SEQUENCE LISTING (52 sequences)

SEQ ID NO: 1 - protein, 976 aa, Homo sapiens [sequence omitted]

SEQ ID NO: 2 - protein, 976 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARVIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - protein, 622 aa, Homo sapiens [sequence omitted]

SEQ ID NO: 4 - protein, 10 aa, Artificial Sequence
  Synthetic
GGSVSSGSYY

SEQ ID NO: 5 - protein, 7 aa, Artificial Sequence
  Synthetic
IYYSGST

SEQ ID NO: 6 - protein, 12 aa, Artificial Sequence
  Synthetic
AREGKNGAFDIW

SEQ ID NO: 7 - protein, 6 aa, Artificial Sequence
  Synthetic
QSISSY

SEQ ID NO: 8 - protein, 4 aa, Artificial Sequence
  Synthetic
AASS

SEQ ID NO: 9 - protein, 10 aa, Artificial Sequence
  Synthetic
QQSYSTPLTF

SEQ ID NO: 10 - protein, 119 aa, Artificial Sequence
  Synthetic
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 11 - protein, 109 aa, Artificial Sequence
  Synthetic
RHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRT

SEQ ID NO: 12 - DNA, 357 bp, Artificial Sequence
  Synthetic
caggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctct

SEQ ID NO: 13 - DNA, 327 bp, Artificial Sequence
  Synthetic
cgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggact

SEQ ID NO: 14 - protein, 243 aa, Artificial Sequence
  Synthetic
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRT

SEQ ID NO: 15 - DNA, 729 bp, Artificial Sequence
  Synthetic
caggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctctggaggtggaggctcaggaggaggaggcagtggaggtggtgggtcacgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggact

SEQ ID NO: 16 - protein, 20 aa, Homo sapiens
METPAQLLFLLLLWLPDTTG

SEQ ID NO: 17 - protein, 20 aa, Mus musculus
METDTLLLWVLLLWVPGSTG

SEQ ID NO: 18 - protein, 15 aa, Artificial Sequence
  Synthetic
GGGGSGGGGSGGGGS

SEQ ID NO: 19 - DNA, 45 bp, Artificial Sequence
  Synthetic
ggaggtggaggctcaggaggaggaggcagtggaggtggtgggtca

SEQ ID NO: 20 - DNA, 45 bp, Artificial Sequence
  Synthetic
ggtggaggcggttcaggcggaggtggctctggcggtggcggatca

SEQ ID NO: 21 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 22 - protein, 247 aa, Homo sapiens
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 23 - protein, 69 aa, Homo sapiens
STTTKPVLRTPSPVHPTGTSQPQRPEDCRPRGSVKGTGLDFACDIYIWAPLAGICVALLLSLIITLICY

SEQ ID NO: 24 - DNA, 207 bp, Homo sapiens
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 25 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 26 - DNA, 81 bp, Homo sapiens
ttttgggtgctggtggtggttggtggagtcctggcttgctatagcttgctagtaacagtggcctttattattttctgggtg

SEQ ID NO: 27 - protein, 27 aa, Homo sapiens
FWVLVVVGGVLACYSLLVTVAFIIFWV

SEQ ID NO: 28 - DNA, 81 bp, Homo sapiens; identical to SEQ ID NO: 26

SEQ ID NO: 29 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 112 aa, Homo sapiens
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 31 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 32 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 33 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 34 - DNA, 63 bp, Mus musculus
aatagtagaaggaacagactccttcaaagtgactacatgaacatgactccccggaggcctggg

SEQ ID NO: 35 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 36 - DNA, 435 bp, Homo sapiens
ccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccag

SEQ ID NO: 37 - DNA, 60 bp, Homo sapiens
atgcagatcccacaggcgccctggccagtcgtctgggcggtgctacaactgggctggcgg

SEQ ID NO: 38 - DNA, 495 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccag

SEQ ID NO: 39 - DNA, 213 bp, Homo sapiens
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 40 - DNA, 219 bp, Homo sapiens
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacagg

SEQ ID NO: 41 - protein, 239 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCN

SEQ ID NO: 42 - DNA, 717 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 43 - protein, 241 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHR

SEQ ID NO: 44 - DNA, 723 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacagg

SEQ ID NO: 45 - protein, 20 aa, Homo sapiens
MYRMQLLSCIALSLALVTNS

SEQ ID NO: 46 - protein, 20 aa, Mus musculus
MYSMQLASCVTLTLVLLVNS

SEQ ID NO: 47 - protein, 20 aa, Homo sapiens
METPAQLLFLLLLWLPDTTG

SEQ ID NO: 48 - protein, 20 aa, Mus musculus
METDTLLLWVLLLWVPGSTG

SEQ ID NO: 49 - protein, 21 aa, Homo sapiens
MALPVTALLLPLALLLHAARP

SEQ ID NO: 50 - protein, 18 aa, Homo sapiens
MALPVTALLLPLALLLHA

SEQ ID NO: 51 - protein, 16 aa, Homo sapiens
MKWVTFISLLFSSAYS

SEQ ID NO: 52 - protein, 30 aa, Homo sapiens
MDSKGSSQKGSRLLLLLVVSNLLLCQGVVS

(19) United States
(12) Patent Application Publication
(10) Pub. No.: US 2023/0051518 A1
(43) Pub. Date: Feb. 16, 2023

(54) CELLS EXPRESSING C-KIT MUTATIONS AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Prasad S. Adusumilli, New York, NY (US); Yuquan Xiong, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/831,638

(22) Filed: Jun. 3, 2022

Related U.S. Application Data
(63) Continuation of application No. PCT/US2020/062992, filed on Dec. 3, 2020.
(60) Provisional application No. 62/943,032, filed on Dec. 3, 2019.

(51) Int. Cl. C07K 14/725; C12N 5/0783; C12N 15/62; C12N 9/12; C07K 14/705; A61P 35/00; A61P 11/00

(52) U.S. Cl. CPC C07K 14/7051; C12N 5/0636; C12N 15/625; C12N 9/12; C12Y 207/10001; C07K 14/70503; A61P 35/00; A61P 11/00; A61K 38/00

(57) ABSTRACT

The present disclosure provides methods and compositions for enhancing the immune response toward cancers and pathogens. The presently disclosed subject matter provides methods and compositions for enhancing the immune response toward cancers and pathogens. It relates to cells comprising a c-Kit mutant, e.g., a c-Kit mutant comprising an activating mutation. The cells can further comprise an antigen-recognizing receptor (e.g., a chimeric antigen receptors (CAR) or a T cell receptors (TCR)). The presently disclosed subject matter relates to the use of cells for treatment, e.g., treating cancers.

### CROSS-REFERENCE TO RELATED APPLICATIONS

The present application is a continuation of International Patent Application No. PCT/US2020/062992 filed Dec. 3, 2020, which claims priority to U.S. Provisional Application No. 62/943,032 filed Dec. 3, 2019, the contents of which are incorporated by reference in their entireties herein, and to which priority is claimed.

### SEQUENCE LISTING

The present application contains a Sequence Listing which has been submitted in ASCII format via EFS-Web and is hereby incorporated by reference in its entirety. Said ASCII copy, created on Jun. 3, 2022, is named 072734_1365_SL and is 57,945 bytes in size.

### 1. INTRODUCTION

The presently disclosed subject matter provides methods and compositions for enhancing the immune response toward cancers and pathogens. It relates to cells comprising a c-Kit mutant, e.g., a c-Kit mutant comprising an activating mutation. The cells can further comprise an antigen-recognizing receptor (e.g., a chimeric antigen receptors (CAR) or a T cell receptors (TCR)). The presently disclosed subject matter relates to the use of cells for treatment, e.g., treating cancers.

### 2. BACKGROUND OF THE INVENTION

Cell-based immunotherapy is a therapy with curative potential for the treatment of cancer. T cells and other immune cells may be modified to target tumor antigens through the introduction of genetic material coding for natural or modified T cell receptors (TCR) or synthetic receptors for antigen, termed Chimeric Antigen Receptors (CARs), specific to selected antigens. Patient-engineered CAR T cells have demonstrated remarkable efficacy against a range of liquid and solid malignancies.

CARs that are in clinic and in preclinical development predominantly use co-stimulatory domains such as CD28 or 4-1BB. Persistence, especially functional persistence of these CARs has shown been to be associated with better comes. There is unmet need for improved CARs having enhanced proliferation and persistence without a co-stimulatory domain, and/or with improved efficiency and activities as compared to the existing CARs.

### 3. SUMMARY OF THE INVENTION

The presently disclosed subject matter provides cells comprising: (a) an antigen-recognizing receptor that binds to an antigen, and (b) a mutant of human c-Kit. In certain embodiments, the c-Kit mutant comprises an activating mutation.

In certain embodiments, the c-Kit is human c-Kit. In certain embodiments, the activating mutation is within the intracellular region of human c-Kit. In certain embodiments, the intracellular region comprises amino acids 544 to 977 of human c-Kit.

In certain embodiments, the activating mutation is within amino acids 816 to 826 of human c-Kit. In certain embodiments, the activating mutation is at amino acid position 816 or amino acid position 822.

In certain embodiments, the activating mutation is within amino acids 550 to 570 of human c-Kit. In certain embodiments, the activating mutation is at amino acid position 560 of human c-Kit.

In certain embodiments, the activating mutation is selected from D816V, D816Y, D816H, D816F, N822K, V560G, or a combination thereof. In certain embodiments, the activating mutation comprises or consists of D816V.

In certain embodiments, the human c-Kit comprises or consists of the amino acid sequence set forth in SEQ ID NO:1.

In certain embodiments, the mutant comprises or consists of the amino acid sequence set forth in SEQ ID NO: 2 or a portion thereof. In certain embodiments, the mutant consists of amino acids 543 to 976 of SEQ ID NO: 2

In certain embodiments, the c-Kit mutant is operably linked to an inducible promoter. In certain embodiments, the inducible promoter is selected from nuclear factor of activated T cells (NFAT), transcriptional response element (TRE) promoter, a CD69 promoter, a CD25 promoter, and an IL-2 promoter.

In certain embodiments, the c-Kit mutant enhances cell persistence of the cell. In certain embodiments, the c-Kit mutant reduces apoptosis or anergy of the cell.

In certain embodiments, the antigen-recognizing receptor is exogenous or endogenous. In certain embodiments, the antigen-recognizing receptor is recombinantly expressed. In certain embodiments, the antigen-recognizing receptor is expressed from a vector. In certain embodiments, the c-Kit mutant is expressed from a vector.

In certain embodiments, the cell is an immunoresponsive cell. In certain embodiments, the cell is a cell of the lymphoid lineage or a cell of the myeloid lineage. In certain embodiments, the cell of the lymphoid lineage is selected from T cells, B cells, Natural Killer (NK) cells, dendritic cells. In certain embodiments, the cell is a T cell. In certain embodiments, the T cell is a cytotoxic T lymphocyte (CTL), a γδ T cell, a tumor-infiltrating lymphocyte (TIL), a regulatory T cell, or a Natural Killer T (NKT) cell.

In certain embodiments, the antigen is a tumor antigen or a pathogen antigen. In certain embodiments, the antigen is a tumor antigen. In certain embodiments, the tumor antigen is selected from the group consisting of mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, ERBB2, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, and CD99, CD70, ADGRE2, CCR1, LILRB2, PRAME, HPV E6 oncoprotein, HPV E7 oncoprotein, and ERBB. In certain embodiments, the antigen is mesothelin.

In certain embodiments, the antigen-recognizing receptor is selected from a T cell receptor (TCR), a chimeric antigen receptor (CAR), and a TCR like fusion molecule. In certain embodiments, the antigen-recognizing receptor is a CAR. In certain embodiments, the CAR comprises an extracellular antigen-binding domain, a transmembrane domain, and an intracellular signaling domain. In certain embodiments, the intracellular signaling domain of the CAR further comprises at least one co-stimulatory signaling region. In certain embodiments, the at least one co-stimulatory signaling region comprises a CD28 polypeptide. In certain embodiments, the CAR does not comprise a co-stimulatory signaling region.

Furthermore, the present disclosure provides methods for producing an antigen-specific cell. In certain embodiments, the method comprises introducing into a cell (a) a first nucleic acid sequence encoding an antigen-recognizing receptor that binds to an antigen; and (b) a second nucleic sequence encoding a c-Kit mutant comprising an activating mutation. In certain embodiments, the first nucleic acid sequence is operably linked to a first promoter. In certain embodiments, the second nucleic acid sequence is operably linked to a second promoter. In certain embodiments, one or both of the first and the second nucleic acid sequences are comprised in a vector. In certain embodiments, the vector is a retroviral vector.

Furthermore, the present disclosure provides compositions comprising: a) a mutant of human c-Kit comprising an activating mutation; and b) an antigen-recognizing receptor that binds to an antigen. In certain embodiments, the -Kit mutant is operably linked to a first promoter. In certain embodiments, the antigen-recognizing receptor is operably linked to a second promoter. In certain embodiments, one or both of the first and second promoters are inducible promoters. In certain embodiments, the inducible promoter is selected from a NFAT transcriptional response element (TRE) promoter, a CD69 promoter, a CD25 promoter, and an IL-2 promoter. The present disclosure further provides cells comprising a composition disclosed herein.

Furthermore, the present disclosure provides nucleic acid compositions comprising (a) a first polynucleotide encoding an antigen-recognizing receptor that binds to an antigen; and (b) a second polynucleotide encoding a mutant of human c-Kit comprising an activating mutation. In certain embodiments, the nucleic acid composition further comprises a first promoter that is operably linked to the c-Kit mutant. In certain embodiments, the nucleic acid composition further comprises a second promoter that is operably linked to the antigen-recognizing receptor. In certain embodiments, one or both of the first and second promoters are inducible promoters. In certain embodiments, the inducible promoter is selected from a NFAT transcriptional response element (TRE) promoter, a CD69 promoter, a CD25 promoter, and an IL-2 promoter. In certain embodiments, one or both of the first and second polynucleotides are comprised in a vector. In certain embodiments, the vector is a retroviral vector. The present disclosure further provides cells comprising a nucleic acid composition disclosed herein.

The present disclosure further provides vectors comprising the nucleic acid composition disclosed herein. The present disclosure further provides cells comprising a vector disclosed herein.

The present disclosure also provides pharmaceutical compositions comprising the cell disclosed herein and a pharmaceutically acceptable excipient. In certain embodiments, the composition further comprises an inhibitor of c-Kit. In certain embodiments, the inhibitor of c-Kit is selected from dasatinib (BMS-354825), midostaurin (PKC412), ponatinib, imatinib, and a combination thereof. In certain embodiments, the pharmaceutical composition is for treating and/or preventing a neoplasm, a pathogen infection, or an infection disease.

The present disclosure further provides methods of reducing tumor burden in a subject. The present disclosure also provides, the method comprises administering to the subject a cell disclosed herein or a pharmaceutical composition disclosed herein. In certain embodiments, the method reduces the number of tumor cells, reduces tumor size, and/or eradicates the tumor in the subject.

The present disclosure further provides methods of treating and/or preventing a neoplasm. In certain embodiments, the method comprises administering to the subject a cell disclosed herein or a pharmaceutical composition disclosed herein.

The present disclosure further provides methods of lengthening survival of a subject having a neoplasia. In certain embodiments, the method comprises administering to the subject a cell disclosed herein or a pharmaceutical composition disclosed herein.

In certain embodiments, the tumor or neoplasm is a solid tumor. In certain embodiments, the solid tumor is selected from the group consisting of mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, cholangiocarcinoma, and a combination thereof. In certain embodiments, the solid tumor is mesothelioma. In certain embodiments, the solid tumor is lung cancer.

In certain embodiments, the method further comprises administering an inhibitor of c-Kit. In certain embodiments, the inhibitor of c-Kit is selected from dasatinib (BMS-354825), midostaurin (PKC412), ponatinib, imatinib, and a combination thereof.

Furthermore, the present disclosure provides kits comprising a cell disclosed herein, a composition disclosed herein, a nucleic acid composition disclosed herein, or a vector disclosed herein. In certain embodiments, the kit further comprises written instructions for treating and/or preventing a neoplasm, a pathogen infection, or an infectious disease.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A and 1B depict a composition in accordance with certain embodiments of the presently disclosed subject matter. The composition shown in FIG. 1A, i.e., “M28z-KITv” (also referred to as M28z-KITm”) comprises a c-Kit mutant D816V and a second generation CAR comprising an anti-mesothelin (MSLN) scFv, a CD28 transmembrane domain, a CD28 cytoplasmic signaling domain, a CD3zeta signaling domain. The composition shown in FIG. 1B, i.e., “Mz-KITv” comprises a c-Kit mutant D816V and a first-generation CAR comprising an anti-mesothelin (MSLN) scFv, a CD28 transmembrane domain, and a CD3zeta signaling domain. LTR represents long terminal repeat.

FIG. 2 depicts transduction efficiency of various constructs to T cells.

FIGS. 3A and 3B depict that M28z-KITv CAR-T constitutively exhibited activated pKIT signaling. FIG. 3A depicts western blot results, which show that M28z-KITv CAR-T exhibited p-KIT activity without SCF, while M28z did not express KIT protein. FIG. 3B depicts that M28z-KITv CAR-T exhibited higher p-STAT3 and p-STAT5 activity than M28z-KITwt control

FIG. 4 depicts cumulative expansion of CAR-T cells during serial coculture. Arrows indicate time points of T-cell re-stimulation with A549GM tumor cells (E:T=3:1).

FIG. 5 depicts enhanced proliferation of M28z CAR T cells and M28z-KITm CAR T cells. Far red cell trace 7 days first antigen stimulation (E:T=2:1). Target cells were A549GM cells.

FIG. 6 depicts proliferation of M28z CAR T cells and M28z-KITm CAR T cells. Far red cell trace 7 days first antigen stimulation (E:T=2:1). Target cells were A549GM cells.

FIGS. 7A and 7B depict cytolytic activity of Mz CAR T cells, M28z CAR T cells, P28z CAR T cells, Mz-KITv CAR T cells, or M28z-KITv CAR T cells in Donor 1. FIG. 7A shows the cytolytic activity at 4 hours. FIG. 7B shows the cytolytic activity at 18 hours. Target cells were high MLSN A549M cells.

FIGS. 8A and 8B depict cytolytic activity of Mz CAR T cells, M28z CAR T cells, Mz-KITv CAR T cells, or M28z-KITv CAR T cells in Donor 2 after two stimulation with target cells (high MLSN A549GM cells) for every 4 day (E:T=3:1). FIG. 8A shows the cytolytic activity at 4 hours post second antigen stimulation. FIG. 8B shows the cytolytic activity at 18 hours post second antigen stimulation.

FIG. 9 depicts cytolytic activity of Mz CAR T cells, M28z CAR T cells, Mz-KITv CAR T cells, or M28z-KITv CAR T cells. The target cells were low MSLN A549G cells.

FIGS. 10A and 10B depict PD1 expression of M28z CART cells, Mz-KITv CAR T cells, or M28z-KITv CAR T cells after antigen stimulation with A549GM cells. FIG. 10A shows CD4⁺ T cells. FIG. 10B shows CD8⁺ T cells.

FIGS. 11A and 11B depict KITv CAR T cells phenotype following antigen stimulation. FIG. 11A depicts exhibition of stem cell-like memory T cells (T_{SCM}) cells of M28z CART cells, Mz-KITv CART cells, or M28z-KITv CAR T cells after stimulation with A546GM cells for every 4 days (E:T=3:1). FIG. 11B depicts released IFN-γ, TNF-α and IL-2 assessed by Luminex assay, after 18 hours co-culture of CAR T cells with MSLN+ cells (E:T=3:1).

FIGS. 12A-12D depict in vivo efficacy of M28z CART cells, Mz-KITv CAR T cells, or M28z-KITv CAR T cells toward low MSLN lung tumor. Mice with established low MSLN A549G lung tumor were treated with a single dose of 1×10⁵ T cells comprising M28z, Mz-KITv or M28z-KITv. UT represents untreated control. FIG. 12A shows the results for UT. FIG. 12B shows the results for M28z CAR T cells. FIG. 12C shows the results for M28z-KITv CAR T cells. FIG. 12D shows the results for Mz-KITv CAR T cells.

FIGS. 13A-13D depict in vivo efficacy of T cells comprising M28z, Mz-KITv or M28z-KITv toward high MSLN lung tumor. Mice with established high MSLN A549GM lung tumor were treated with a single dose of 1×10⁵ T cells comprising M28z, Mz-KITv or M28z-KITv. UT represents untreated control. FIG. 13A shows the results for UT. FIG. 13B shows the results for M28z CAR T cells. FIG. 13C shows the results for M28z-KITv CAR T cells. FIG. 13D shows the results for Mz-KITv CAR T cells.

FIGS. 14A-14C depict Kaplan-Meier survival curves for in vivo treated mice depicted in FIGS. 12A-12D and 13A-13D. FIG. 14A depicts the survival curve of mice with established low-antigen (mesothelin) expressing lung tumors. FIG. 14 B depicts the survival curve of mice with established high-antigen (mesothelin) expressing lung tumors. FIG. 14C depicts the FACS measurements of mesothelin expression levels in low mesothelin-expressing lung cancer (A549G) and high mesothelin-expressing lung cancer (A549GM).

FIG. 15 depicts sensitivity of M28z CART cells and M28z-KITv CART cells to clinical tyrosine kinase inhibitors.

FIGS. 16A-16C depict the anti-tumor activity of M28z, Mz-KITv or M28z-KITv CAR T cells towards high MSLN-expressing lung tumor. NSG Mice with established high MSLN A549GM lung tumor were treated with a single dose of 1×10⁵ M28z, M28z-KITv or Mz-KITv CAR T cells. UT: untreated control. FIG. 16A depicts the results of in vivo monitoring of tumor burden in mice using bioluminescent imaging (BLI). FIGS. 16B and 16C depict the Kaplan-Meier survival analysis of the mice, indicating the in vivo efficacy of i.v. administration of different CART cells. *, P<0.05. ***, P<0.001.

FIGS. 17A-17C depict the anti-tumor activity of M28z, Mz-KITv and M28z-KITv CAR T cells towards low MSLN-expressing lung tumor. NSG Mice with established low MSLN A549G lung tumor were treated with a single dose of 1×10⁵ M28z, M28z-KITv or Mz-KITv CART cells. UT: untreated control. FIG. 17A depicts the results of in vivo monitoring of tumor burden in mice using bioluminescent imaging (BLI). FIGS. 17B and 17C depict the Kaplan-Meier survival analysis of the mice, indicating the in vivo efficacy of i.v. administration of different CART cells. **, P<0.01.

FIGS. 18A and 18B depict Kaplan-Meier survival analysis that compared the in vivo efficacy of intrapleural administration of M28z, Mz-KITv or M28z-KITv CAR T cells in pleural mesothelioma tumor model. NSG Mice with established high MSLN MGM mesothelioma were treated with a single dose of 5×10⁴ P28z, Mz, M28z, M28z-KITv and Mz-KITv CAR T cells. *, P<0.05. FIG. 18A depicts the comparison among all groups. FIG. 18B depicts the comparison between M28z and M28z-KITv, and between Mz and Mz-KITv groups.

FIGS. 19A and 19B depict the anti-tumor activity of M28z, Mz-KITv and M28z-KITv CAR T cells towards low MSLN-expressing mesothelioma. FIG. 19A depicts the expression of low or high MSLN protein in MSTO cell to produce MG-LM and MGM cell respectively. FIG. 19B depicts NSG Mice with established low MSLN mesothelioma (MG-LM) were treated with a single dose of 5×10⁴ P28z, M28z, M28z-KITv or Mz-KITv CAR T cells. Kaplan-Meier survival analysis compared the in vivo efficacy of intrapleural administration of different CART cells. *, P<0.05. **, P<0.01.

FIG. 20 depicts p-ERK signal of CAR T cells after antigen stimulation. After co-culture with MGM cell (E:T=1:2) for 5 min, p-ERK levels of CD4+ and CD8+ CAR T cell were measured by FACS. Both CD4 and CD8 of CART cells of M28z-KITv and Mz-KITv had stronger p-ERK activity than M28z.

FIGS. 21A and 21B depict that CAR T cells obtained from mice were exposed to high mesothelin-expressing mesothelioma cells and were analyzed for PD1 expression. FIG. 21A depicts the quantification of PD1 expression at different E:T ratios. FIG. 21B depicts the flow cytometry graph measuring PD1 expression at different E:T ratios.

FIGS. 22A and 22B depict enriched gene sets related to phenotypic and functional T cell features in M28z-KITv CAR T cells. After co-culture with MSLN⁺ tumor cells for 24 hours, CD8 CAR T cells of M28z and M28z-KITv were collected for analysis by nano string for CAR T panel genes, n=3 for each group. FIG. 22A depicts that 87 out of 780 detected genes had significant fold change. FIG. 22B depicts a heatmap of Pathway scores showing enriched gene sets related to phenotypic and functional T cell features in M28z-KITv CAR T cells. FIG. 22C provides the gene pathways that were upregulated in KIT CAR T cells.

FIGS. 23A-23B depict significant upregulation of interferon signaling genes in M28z-KITv CAR T cells compared to M28z CAR T cells. After co-cultured with MSLN+ cancer cells for 24 hours, CD8 CART cells of M28z and M28z-KITv were collected for analysis by nano string for CAR T panel genes, n=3 for each group. The expression of Type I interferon signaling genes (FIG. 23A) and Type II interferon signaling genes (FIG. 23B) significantly increased in M28z-KITv CAR T cells.

### 5. DETAILED DESCRIPTION OF THE INVENTION

The presently disclosed subject matter provides cells comprising a c-Kit mutant, wherein the c-Kit mutant comprises an activating mutation. The cells can be genetically modified immunoresponsive cells (e.g., T cells or NK cells), cells comprise an antigen-recognizing receptor (e.g., a T-cell receptor (TCR) or a chimeric antigen receptor (CAR)). The presently disclosed subject matter also provides methods of using such cells for inducing and/or enhancing an immune response to a target antigen, and/or for treating and/or preventing a neoplasia, a pathogen infection, or other diseases/disorders (e.g., a disease/disorder where an increase in an antigen-specific immune response is desired). The presently disclosed subject matter is based, at least in part, on the discovery that a c-Kit mutant (e.g., c-KitD816V) can enhance the cell proliferation of cells (e.g., T cells) comprising an antigen-recognizing receptor (e.g., a CAR).

Non-limiting embodiments of the present disclosure are described by the present specification and Examples.

For purposes of clarity of disclosure and not by way of limitation, the detailed description is divided into the following subsections:
5.1. Definitions;
5.2. c-Kit Mutants;
5.3. Cells;
5.4. Antigen-Recognizing Receptors;
5.5. Dominant Negative Form of Programmed Death 1 (PD-1 DN)
5.6. Compositions and Vectors;
5.7. Polypeptides and Analogs;
5.8. Administration;
5.9. Formulations;
5.10. Methods of Treatment; and
5.11. Kits

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)₂, and Fab fragments that lack the Fe fragment of intact antibody, clear more rapidly from the circulation, and may have less non-specific tissue binding of an intact antibody (Wahl et al., J Nucl Med (1983); 24:316-325). As used herein, antibodies include whole native antibodies, bispecific antibodies; chimeric antibodies; Fab, Fab′, single chain variable fragments (scFvs), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat et al., Sequences of Proteins of Immunological Interest, Fifth Edition, U.S. Department of Health and Human Services (1991); NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker is usually rich in glycine for flexibility, as well as serine or threonine for solubility. “Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in SEQ ID NO: 16, which is provided below:

[SEQ ID NO: 16]
GGGGSGGGGSGGGGS.

In certain embodiments, the nucleotide sequence encoding the amino acid sequence of SEQ ID NO: 16 is set forth in SEQ ID NO: 17, which is provided below:

[SEQ ID NO: 17]
GGAGGTGGAGGCTCAGGAGGAGGAGGCAGTGGAGGTGGTGGGTCA.

In certain embodiments, the nucleotide sequence encoding the amino acid sequence f SEQ ID NO: 16 is set forth in SEQ ID NO: 18, which is provided below.

[SEQ ID NO: 18]
GGTGGAGGCGGTTCAGGCGGAGGTGGCTCTGGCGGTGGCGGATCA

Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston et al., Proc Nat Acad Sci USA (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (Zhao et al., Hyrbidoma (Larchmt) 2008; 27(6):455-51; Peter et al., J Cachexia Sarcopenia Muscle (2013); 4(1):79-86; Shieh et al., J Imunol (2009); 183(4):2277-85; Giomarelli et al., Thromb Haemost (2007); 97(6):955-63; Fife et al., JCI (2006); 116(8):2252-61; Brocks et al., Immunotechnology (1997); 3(3):173-84; Moosmayer et al., Ther Immunol (1995); 2(10):31-40). Agonistic scFvs having stimulatory activity have been described (Peter et al., J Biol Chem (2003); 25278(38):36740-7; Xie et al., Nat Biotech (1997); 15(8):768-71; Ledbetter et al., Crit Rev Immunol (1997); 17(5-6):427-55; Ho et al., BioChem Biophys Acta (2003); 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest. Such nucleic acid molecules need not to be 100% homologous or identical with an endogenous nucleic acid sequence, but may exhibit substantial identity.

Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (Wahl et al., Methods Enzymol (1987); 152:399; Kimmel, Methods Enzymol (1987); 152:507).

For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described (Benton et al., Science (1977); 196:180; Grunstein et al., Proc Natl Acad Sci, USA (1975); 72:3961; Ausubel et al., Current Protocols in Molecular Biology (2001); Wiley Interscience, New York; Berger et al., Guide to Molecular Cloning Techniques (1987); Academic Press, New York); and Sambrook et al., Molecular Cloning: A Laboratory Manual, (1987); Cold Spring Harbor Laboratory Press, New York).

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasia, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasia.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). In certain embodiments, the neoplasm is a cancer. In certain embodiments, the neoplasm is a solid tumor.

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 43] (human), MYSMQLASCVTLTLVLLVNS [SEQ ID NO: 44] (mouse); the kappa leader sequence: METPAQLLFLLLLWLPDTTG [SEQ ID NO: 45] (human), METDTLLLWVLLLWVPGSTG [SEQ ID NO: 46] (mouse); the CD8 leader sequence: MALPVTALLLPLALLLHAARP [SEQ ID NO: 47] (human); the truncated human CD8 signal peptide: MALPVTALLLPLALLLHA [SEQ ID NO: 48] (human); the albumin signal sequence: MKWVTFISLLFSSAYS [SEQ ID NO: 49] (human); and the prolactin signal sequence: MDSKGSSQKGSRLLLLLVVSNLLLCQGVVS [SEQ ID NO: 20] (human). By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

5.2. c-Kit Mutants

Proto-oncogene KIT is a receptor tyrosine kinase protein and also known as CD117; KIT; PBT; stem cell growth factor receptor (SCFR); MASTC. GenBank ID: 3815 (human), 16590 (mouse). The protein product of KIT includes, but is not limited to, NCBI Reference Sequences NP_000213 (human isoform 1), NP_001122733 (mouse isoform 1).

c-Kit, known as CD117, is a cytokine receptor expressed on the surface of hematopoietic stem cells as well as other cell types. Signaling through c-Kit plays a role in cell survival, proliferation and differentiation (Ceredig et al., Nat Rev Immunol (2002); 2(11):888-97).

c-Kit binds to stem cell factor (SCF). Upon binding, c-Kit and SCF form a dimer that activates its intrinsic tyrosine kinase activity and in turn phosphorylates and activates signal transduction molecules that propagate the signal in the cell. A presently disclosed c-Kit activating mutation (e.g., D816V mutation) results in constitutive activation absent SCF, e.g., without forming the cKit/SCF dimers (Hirota et al., Science (1998); 279(5350):577-80; Kitamura et al., Mut Res (2001):165-71).

In certain embodiments, a c-Kit mutant is a human c-Kit mutant. In certain embodiments, a human c-Kit protein comprises or consists of the sequence having a NCBI Reference No. NP_000213 (SEQ ID NO: 1). SEQ ID NO: 1 is provided below.

| [SEQ ID NO: 1] | |
|---|---|
| 1 | MRGARGAWDE LCVLLLLLRV QTGSSQPSVS PGEPSPPSIH PGKSDLIVRV GDEIRLLCTD |
| 61 | PGFVKWTFEI LDETNENKQN EWITEKAEAT NTGKYTCTNK HGLSNSIYVF VRDPAKLFLV |
| 121 | DRSLYGKEDN DTLVRCPLTD PEVTNYSLKG CQGKPLPKDL RFIPDPKAGI MIKSVKRAYH |
| 181 | RLCLHCSVDQ EGKSVLSEKF ILKVRPAFKA VPVVSVSKAS YLLREGEEFT VTCTIKDVSS |
| 241 | SVYSTWKREN SQTKLQEKYN SWHHGDFNYE RQATLTISSA RVNDSGVFMC YANNTFGSAN |
| 301 | VTTTLEVVDK GFINIFPMIN TTVFVNDGEN VDLIVEYEAF PKPEHQQWIY MNRTFTDKWE |
| 361 | DYPKSENESN IRYVSELHLT RLKGTEGGTY TFLVSNSDVN AAIAFNVYVN TKPEILTYDR |
| 421 | LVNGMLQCVA AGFPEPTIDW YFCPGTEQRC SASVLPVDVQ TLNSSGPPFG KLVVQSSIDS |
| 481 | SAFKHNGTVE CKAYNDVGKT SAYFNFAFKG NNKEQIHPHT LFTPLLIGFV IVAGMMCIIV |
| 541 | MILTYKYLQK PMYEVQWKVV EEINGNNYVY IDPTQLPYDH KWEFPRNRLS FGKTLGAGAF |
| 601 | GKVVEATAYG LIKSDAAMTV AVKMLKPSAH LTEREALMSE LKVLSYLGNH MNIVNLLGAC |
| 661 | TIGGPTLVIT EYCCYGDLLN FLRRKRDSFI CSKQEDHAEA ALYKNLLHSK ESSCSDSTNE |
| 721 | YMDMKPGVSY VVPTKADKRR SVRIGSYIER DVTPAIMEDD ELALDLEDLL SFSYQVAKGM |
| 781 | AFLASKNCIH RDLAARNILL THGRITKICD FGLARDIKND SNYVVKGNAR LPVKWMAPES |
| 841 | IFNCVYTFES DVWSYGIFLW ELFSLGSSPY PGMPVDSKFY KMIKEGFRML SPEHAPAEMY |
| 901 | DIMKTCWDAD PLKRPTFKQI VQLIEKQISE STNHIYSNLA NCSPNRQKPV VDHSVRINSV |
| 961 | GSTASSSQPL LVHDDV |

The cells of the presently disclosed subject matter comprise a c-Kit mutant. In certain embodiments, the c-Kit mutant comprises an activating mutation.

In certain embodiments, the activating mutation is a gain-of-function mutation. In certain embodiments, the activating mutation is a mutation whose gene product consists of an enhanced effect as compared to a gene product without such mutation (e.g., the wild-type protein).

In certain embodiments, the activating mutation (e.g., D816V mutation) is present in early lineage of hemopoietic cells, and is lost during maturation. In certain embodiments, the activating mutation of c-Kit leads to c-Kit activation independent of the interaction of the c-Kit and its ligand (e.g., SCF). In certain embodiments, the activating mutation of c-Kit leads to c-Kit activation absent a c-Kit ligand, e.g., SCF. In certain embodiments, the activating mutation of c-Kit leads to constitutive activation of c-Kit. In certain embodiments, the activating mutation of c-Kit leads to c-Kit activation independent of the inhibition of Tyrosine phosphatase-1 (SHP-1) and/or Tyrosine phosphatase-2 (SHP-2). In certain embodiments, the activating mutation of c-Kit leads to c-Kit activation in the presence of inhibition of SHP-1 and/or SHP-2.

The activating mutation of c-Kit promotes proliferation and anti-apoptosis of cells comprising the c-Kit mutant. The activating mutation of c-Kit makes cells comprising the c-Kit mutant resistant to PD-L1/2-PD-1 inhibition.

In certain embodiments, the activating mutation is within the intracellular region of human cKIT, e.g., a human cKIT consisting of the amino acid sequence set forth in SEQ ID NO: 1. In certain embodiments, the intracellular region of human cKIT comprises amino acids 544 to 977 of SEQ ID NO: 1. In certain embodiments, the activating mutation is within amino acids 816 to 826 of human cKIT, e.g., a human cKIT consisting of the amino acid sequence set forth in SEQ ID NO: 1. In certain embodiments, the activating mutation is at amino acid position 816, or amino acid position 822. In certain embodiments, the activating mutation is within amino acids 550 to 570 of human cKIT, e.g., a human cKIT consisting of the amino acid sequence set forth in SEQ ID NO: 1. In certain embodiments, the activating mutation is at amino acid position 560. Non-limiting examples of c-Kit activating mutation include D816V, D816Y, D816H, D816F, N822K, V560G, or a combination thereof. In certain embodiments, the activating mutation is D816V.

The c-Kit mutant can be operably linked to a promoter. The promoters can be endogenous or exogenous. Non-limiting examples of exogenous promoters include an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. Non-limiting examples of inducible promoter are selected from a NFAT transcriptional response element (TRE) promoter, a CD69 promoter, a CD25 promoter, and an IL-2 promoter. The inducible promoter can control the activation of c-Kit, e.g., with the control of the inducible promoter, c-Kit is activated only upon the activation of the cells comprising the c-Kit mutant (e.g., T cells or CAR-T cells).

In certain embodiments, the c-Kit mutant comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 2 or a portion thereof. In certain embodiments, the c-Kit mutant comprises or consists of the amino acid sequence set forth in SEQ ID NO: 2 or a portion thereof. In certain embodiments, the c-Kit mutant comprises or consists of an amino acid sequence that is a consecutive portion of SEQ ID NO: 2, which is at least 50, or at least 100, or at least 150, or at least 200, or at least 250, or at least 300, or at least 350, or at least 400, or at least 450, or at least 500, or at least 550, or at least 600, or at least 650, or at least 700, or at least 750, or at least 800, or at least 850, or at least 900, or at least 950, and up to 976 amino acids in length. Alternatively or additionally, in non-limiting various embodiments, the c-Kit mutant comprises or consists of an amino acid sequence of amino acids 1 to 976, 1 to 200, 400 to 976, 500 to 976, or 543 to 976 of SEQ ID NO: 2. In certain embodiments, the c-Kit mutant comprises or consists of amino acids 543 to 976 of SEQ ID NO: 2.

SEQ ID NO: 2 is provided below.

| [SEQ ID NO: 2] | |
|---|---|
| 1 | MRGARGAWDE LCVLLLLLRV QTGSSQPSVS PGEPSPPSIH PGKSDLIVRV GDEIRLLCTD |
| 61 | PGFVKWTFEI LDETNENKQN EWITEKAEAT NTGKYTCTNK HGLSNSIYVF VRDPAKLFLV |
| 121 | DRSLYGKEDN DTLVRCPLTD PEVTNYSLKG CQGKPLPKDL RFIPDPKAGI MIKSVKRAYH |
| 181 | RLCLHCSVDQ EGKSVLSEKF ILKVRPAFKA VPVVSVSKAS YLLREGEEFT VTCTIKDVSS |
| 241 | SVYSTWKREN SQTKLQEKYN SWHHGDFNYE RQATLTISSA RVNDSGVFMC YANNTEGSAN |
| 301 | VTTTLEVVDK GFINIFPMIN TTVFVNDGEN VDLIVEYEAF PKPEHQQWIY MNRTFTDKWE |
| 361 | DYPKSENESN IRYVSELHLT RLKGTEGGTY TFLVSNSDVN AAIAFNVYVN TKPEILTYDR |
| 421 | LVNGMLQCVA AGFPEPTIDW YFCPGTEQRC SASVLPVDVQ TLNSSGPPFG KLVVQSSIDS |
| 481 | SAFKHNGTVE CKAYNDVGKT SAYFNFAFKG NNKEQIHPHT LFTPLLIGFV IVAGMMCIIV |
| 541 | MILTYKYLQK PMYEVQWKVV EEINGNNYVY IDPTQLPYDH KWEFPRNRLS FGKTLGAGAF |
| 601 | GKVVEATAYG LIKSDAAMTV AVKMLKPSAH LTEREALMSE LKVLSYLGNH MNIVNLLGAC |
| 661 | TIGGPTLVIT EYCCYGDLLN FLRRKRDSFI CSKQEDHAEA ALYKNLLHSK ESSCSDSTNE |
| 721 | YMDMKPGVSY VVPTKADKRR SVRIGSYIER DVTPAIMEDD ELALDLEDLL SFSYQVAKGM |
| 781 | AFLASKNCIH RDLAARNILL THGRITKICD FGLARVIKND SNYVVKGNAR LPVKWMAPES |
| 841 | IFNCVYTFES DVWSYGIFLW ELFSLGSSPY PGMPVDSKFY KMIKEGFRML SPEHAPAEMY |
| 901 | DIMKTCWDAD PLKRPTFKQI VQLIEKQISE STNHIYSNLA NCSPNRQKPV VDHSVRINSV |
| 961 | GSTASSSQPL LVHDDV |

### 5.3. Cells

The presently disclosed subject matter provides cells comprising a c-Kit mutant disclosed herein (e.g., one disclosed in Section 5.2). In certain embodiments, the c-Kit mutant is an exogenous c-Kit mutant.

In certain embodiments, the cell is selected from cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells. In certain embodiments, the cell is allogeneic.

The cells of the presently disclosed subject matter can be cells of the myeloid lineage. Non-limiting examples of cells of the myeloid lineage include monocytes, macrophages, basophils, neutrophils, eosinophils, megakaryocytes, mast cell, erythrocyte, thrombocytes, and stem cells from which myeloid cells may be differentiated.

In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed cells have increased cell proliferation, and/or cell persistence. In certain embodiments, the presently disclosed immunoresponsive cells have decreased apoptosis and/or anergy.

In certain embodiments, the cell further comprises an antigen-recognizing receptor (e.g., a CAR or a TCR) that binds to an antigen. The cells can be transduced with the antigen-recognizing receptor and the c-Kit mutant exogenous activating such that the cells co-express the antigen-recognizing receptor and the c-Kit mutant.

The c-Kit mutant can be operably linked to a first promoter. The antigen-recognizing receptor can be operably linked to a second promoter. The first promoter can be the same as the second promoter. Alternatively, the first promoter is different from the second promoter. The first and the second promoters can be endogenous or exogenous. Non-limiting examples of exogenous promoters include an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, one or both of the first and second promoters are inducible promoters. Non-limiting examples of inducible promoter are selected from a NFAT transcriptional response element (TRE) promoter, a CD69 promoter, a CD25 promoter, and an IL-2 promoter.

### 5.4. Antigen-Recognizing Receptors

In certain embodiments, a presently disclosed cell further comprises an antigen-recognizing receptor. In certain embodiments, the antigen-recognizing receptors binds to an antigen. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule.

5.4.1. Antigens

The antigen-recognizing receptor can bind to a tumor antigen or a pathogen antigen.

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. In certain embodiments, the antigen is expressed in a tumor tissue. Non-limiting examples of tumor antigens include Mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-a2, chain, KDR, LeY, L1 cell adhesion molecule, MAGE-A1, ERBB2, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, HPV E6 oncoprotein, HPV E7 oncoprotein, and ERBB. In certain embodiments, the tumor antigen is mesothelin.

In certain embodiments, the antigen-recognizing receptor binds to mesothelin. In certain embodiments, the antigen-recognizing receptor binds to human mesothelin consisting of the sequence with a NCBI Reference No: AAV87530.1 (SEQ ID NO: 3), or fragments thereof. SEQ ID NO: 3 is provided below:

[SEQ ID NO: 3]
MALPTARPLL GSCGTPALGS LLFLLFSLGW VQPSRTLAGE
TGQEAAPLDG VLANPPNISS LSPRQLLGFP CAEVSGLSTE
RVRELAVALA QKNVKLSTEQ LRCLAHRLSE PPEDLDALPL
DLLLFLNPDA FSGPQACTHF FSRITKANVD LLPRGAPERQ
RLLPAALACW GVRGSLLSEA DVRALGGLAC DLPGRFVAES
AEVLLPRLVS CPGPLDQDQQ EAARAALQGG GPPYGPPSTW
SVSTMDALRG LLPVLGQPII RSIPQGIVAA WRQRSSRDPS
WRQPERTILR PRFRREVEKT ACPSGKKARE IDESLIFYKK
WELEACVDAA LLATQMDRVN AIPFTYEQLD VLKHKLDELY
PQGYPESVIQ HLGYLFLKMS PEDIRKWNVT SLETLKALLE
VNKGHEMSPQ VATLIDRFVK GRGQLDKDTL DTLTAFYPGY
LCSLSPEELS SVPPSSIWAV RPQDLDTCDP RQLDVLYPKA
RLAFQNMNGS EYFVKIQSFL GGAPTEDLKA LSQQNVSMDL
ATFMKLRTDA VLPLTVAEVQ KLLGPHVEGL KAEERHRPVR
DWILRQRQDD LDTLGLGLQG GIPNGYLVLD LSVQEALSGT
PCLLGPGPVL TVLALLLAST LA

In certain embodiments, the antigen-recognizing receptor binds to a pathogen antigen, e.g., for use in treating and/or preventing a pathogen infection or other infectious disease, for example, in an immunocompromised subject. Non-limiting examples of pathogens include a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Caliciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses), human papilloma virus (i.e. HPV), JC virus, Epstein Bar Virus, Merkel cell polyoma virus.

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

5.4.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR found on the surface of T cells is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each consists of three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

5.4.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First-generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule. In certain embodiments, the antigen-recognizing receptor is a second-generation CAR.

In certain embodiments, the CAR can comprise an extracellular antigen-binding domain, a transmembrane domain, and an intracellular signaling domain, wherein the extracellular antigen-binding domain specifically binds to an antigen, which can be a tumor antigen or a pathogen antigen.

5.4.3.1. Extracellular Antigen-Binding Domain of a CAR

In certain embodiments, the extracellular antigen-binding domain specifically binds to an antigen. In certain embodiments, the antigen is mesothelin. In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein.

In certain non-limiting embodiments, the extracellular antigen-binding domain of the CAR (embodied, for example, an scFv or an analog thereof) binds to an antigen with a dissociation constant (K_{d}) of about 2×10⁻⁷ M or less. In certain embodiments, the K_{d} is about 2×10⁻⁷M or less, about 1×10⁻⁷ M or less, about 9×10⁻⁸ M or less, about 1×10⁻⁸ M or less, about 9×10⁻⁹M or less, about 5×10⁻⁹M or less, about 4×10⁻⁹ M or less, about 3×10⁻⁹ or less, about 2×10⁻⁹ M or less, or about 1×10⁻⁹ M or less. In certain non-limiting embodiments, the K_{d} is about 3×10⁻⁹M or less. In certain non-limiting embodiments, the K_{d} is from about 1×10⁻⁹ M to about 3×10⁻⁷ M. In certain non-limiting embodiments, the K_{d} is from about 1.5×10⁻⁹M to about 3×10⁻⁷M. In certain non-limiting embodiments, the K_{d} is from about 1.5×10⁻⁹ M to about 2.7×10⁻⁷ M.

In certain non-limiting embodiments, the extracellular antigen-binding domain of the CAR has a high binding specificity as well as high binding affinity to human mesothelin. For example, in such embodiments, the extracellular antigen-binding domain of the CAR (embodied, for example, in an scFv) binds to human mesothelin with an EC50 value of from about 1 nM to about 25 nM as measured by enzyme-linked immunosorbent assay (ELISA). In certain embodiments, the extracellular antigen-binding domain of the CAR has an EC50 value of about 20 nM as measured by ELISA. In certain embodiments, the extracellular antigen-binding domain of the CAR comprises an anti-mesothelin antibody or an antigen-binding portion thereof described in U.S. Pat. No. 8,357,783, which is herein incorporated by reference in its entirety. In certain embodiments, the extracellular antigen-binding domain of the CAR is derived from a heavy chain variable region and a light chain variable region of an antibody that binds to human mesothelin, e.g., antibody m912 as disclosed in Feng et al., Mol. Cancer Therapy (2009); 8(5):1113-1118, which is herein incorporated by reference in its entirety. Antibody m912 was isolated from a human Fab library by panning against recombinant mesothelin. In certain embodiments, the extracellular antigen-binding domain of the CAR is derived from Fab's (e.g., from human or mouse Fab libraries).

Binding of the extracellular antigen-binding domain (embodiment, for example, in an scFv) of the CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the mesothelin targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In one embodiment, the mesothelin-targeted human scFv is labeled with GFP.

In certain non-limiting embodiments, the extracellular antigen-binding domain of the CAR recognizes or binds to human mesothelin with a mesothelin level of about 1,000 or more mesothelin binding sites/cell. In certain embodiments, the extracellular antigen-binding domain of the CAR recognizes or binds to human mesothelin with a mesothelin level of from about 1,000 to about 50,000 mesothelin binding sites/cell. In some embodiments, the extracellular antigen-binding domain of the CAR does not recognize or bind to human mesothelin with a mesothelin expression level of less than 1,000 mesothelin binding sites/cell, e.g., the human mesothelin expressed in normal tissues, e.g., normal pleura, pericardium, and peritoneum tissues. In certain embodiments, the extracellular antigen-binding domain of the CAR does not recognize or bind to human mesothelin with a mesothelin expression level of more than 50,000 mesothelin binding sites/cell. In certain embodiments, a human scFv comprised in the CAR recognizes or binds to human mesothelin with a mesothelin expression level of from about 1,000 to about 50,000 mesothelin binding sites/cell. In certain embodiments, a human scFv comprised in the CAR does not recognize or bind to human mesothelin with a mesothelin expression level of more than 50,000 or less than 1,000 mesothelin binding sites/cell.

In certain embodiments, the extracellular antigen-binding domain of the CAR (e.g., an scFv) comprises a V_{H} CDR1 comprising the amino acid sequence set forth in SEQ ID NO: 4, or a conservative modification thereof, a V_{H} CDR2 comprising the amino acid sequence set forth in SEQ ID NO: 5 or a conservative modification thereof, and a V_{H} CDR3 comprising the amino acid sequence set forth in SEQ ID NO: 6, a conservative modification thereof. In certain embodiments, the extracellular antigen-binding domain of the CAR (e.g., an scFv) comprises a V_{H} CDR1 comprising the amino acid sequence set forth in SEQ ID NO: 4, a V_{H} CDR2 comprising the amino acid sequence set forth in SEQ ID NO: 5, and a V_{H} CDR3 comprising the amino acid sequence set forth in SEQ ID NO: 6. In certain embodiments, the extracellular antigen-binding domain of the CAR (e.g., a scFv) comprises a V_{L} CDR1 comprising the amino acid sequence set forth in SEQ ID NO: 7 or a conservative modification thereof, a V_{L} CDR2 comprising the amino acid sequence set forth in SEQ ID NO: 8 or a conservative modification thereof, and a V_{L} CDR3 comprising the amino acid sequence set forth in SEQ ID NO: 9 or a conservative modification thereof. In certain embodiments, the extracellular antigen-binding domain of the CAR (e.g., a scFv) comprises a V_{L} CDR1 comprising the amino acid sequence set forth in SEQ ID NO: 7, a V_{L} CDR2 comprising the amino acid sequence set forth in SEQ ID NO: 8, and a V_{L} CDR3 comprising the amino acid sequence set forth in SEQ ID NO: 9.

In certain embodiments, the extracellular antigen-binding domain of the CAR (e.g., an scFv) comprises a V_{H} CDR1 comprising the amino acid sequence set forth in SEQ ID NO: 4 or a conservative modification thereof, a V_{H} CDR2 comprising the amino acid sequence set forth in SEQ ID NO: 5 or a conservative modification thereof, a V_{H} CDR3 comprising the amino acid sequence set forth in SEQ ID NO: 6, a conservative modification thereof, a V_{L} CDR1 comprising the amino acid sequence set forth in SEQ ID NO: 7 or a conservative modification thereof, a V_{L} CDR2 comprising the amino acid sequence set forth in SEQ ID NO: 8 or a conservative modification thereof, and a V_{L} CDR3 comprising the amino acid sequence set forth in SEQ ID NO: 9 or a conservative modification thereof. In certain embodiments, the extracellular antigen-binding domain comprises a V_{H} CDR1 comprising amino acids having the sequence set forth in SEQ ID NO: 4, a V_{H} CDR2 comprising the amino acid sequence set forth in SEQ ID NO: 5, a V_{H} CDR3 comprising the amino acid sequence set forth in SEQ ID NO: 6, a V_{L} CDR1 comprising the amino acid sequence set forth in SEQ ID NO: 7, a V_{L} CDR2 comprising the amino acid sequence set forth in SEQ ID NO: 8 and a V_{L} CDR3 comprising the amino acid sequence set forth in SEQ ID NO: 9. In certain embodiments, the CDRs are identified according to the Kabat numbering system.

In certain embodiments, the extracellular antigen-binding domain of the CAR (e.g., an scFv) comprises a heavy chain variable region (V_{H}) comprising the amino acid sequence set forth in SEQ ID NO: 10. In certain embodiments, the extracellular antigen-binding domain of the CAR (e.g., an scFv) comprises a light chain variable region (V_{L}) comprising the amino acid sequence set forth in SEQ ID NO: 11. In certain embodiments, the extracellular antigen-binding domain of the CAR (e.g., a scFv) comprises a V_{H} comprising the amino acid sequence set forth in SEQ ID NO: 10, and a V_{L} comprising the amino acid sequence set forth in SEQ ID NO: 11, optionally with (iii) a linker sequence, for example a linker peptide, between the V_{H} and the V_{L}. In certain embodiments, the linker comprises amino acids consisting of the amino acid sequence set forth in SEQ ID NO: 16. In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to SEQ ID NO: 10. For example, the extracellular antigen-binding domain of the CAR (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 10. In certain embodiments, the extracellular antigen-binding domain comprises a V_{H} comprising the amino sequence set forth in SEQ ID NO: 10. In certain embodiments, the extracellular antigen-binding domain of the CAR (e.g., an scFv) comprises a V_{L} comprising an amino acid sequence that is at least about 80% (e.g., at least about 85%, at least about 90%, or at least about 95%) homologous or identical to SEQ ID NO: 11. For example, the extracellular antigen-binding domain of the CAR (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 11. In certain embodiments, the extracellular antigen-binding domain of the CAR (e.g., an scFv) comprises a V_{L} comprising the amino acid sequence set forth in SEQ ID NO: 11. In certain embodiments, the extracellular antigen-binding domain of the CAR (e.g., an scFv) comprises a V_{H} comprising an amino acid sequence that is at least about 80% (e.g., at least about 85%, at least about 90%, or at least about 95%) homologous or identical to SEQ ID NO: 10, and a V_{L} comprising an amino acid sequence that is at least about 80% (e.g., at least about 85%, at least about 90%, or at least about 95%) homologous or identical to SEQ ID NO: 11. In certain embodiments, the extracellular antigen-binding domain of the CAR (e.g., an scFv) comprises a V_{H} comprising the amino acid sequence set forth in SEQ ID NO: 10 and a V_{L} comprising the amino acid sequence set forth in SEQ ID NO: 11.

An exemplary nucleic acid sequence encoding the amino acid sequence of SEQ ID NO: 10 is set forth in SEQ ID NO: 12.

An exemplary nucleic acid sequence encoding the amino acid sequence of SEQ ID NO: 11 is set forth in SEQ ID NO: 13.

In certain embodiments, the extracellular antigen-binding domain of the CAR (e.g., an scFv) comprises an amino acid sequence that is at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homologous or identical to the amino acid sequence set forth in SEQ ID NO: 14. In certain embodiments, the extracellular antigen-binding domain of the CAR (e.g., an scFv) comprises or consists of the amino acid sequence set forth in SEQ ID NO: 14. In certain embodiments, the extracellular antigen-binding domain of the CAR (e.g., an scFv) specifically binds to a human mesothelin polypeptide (e.g., a human mesothelin polypeptide comprising the amino acid sequence set forth in SEQ ID NO: 3).

In certain embodiments, an exemplary nucleotide sequence encoding the amino acid sequence of SEQ ID NO: 14 is set forth in SEQ ID NO: 15.

In certain embodiments, the scFv is a human scFv.

SEQ ID Nos: 4-15 are provided below:

[SEQ ID NO: 4]
GGSVSSGSYY
[SEQ ID NO: 5]
IYYSGST
[SEQ ID NO: 6]
AREGKNGAFDIW
[SEQ ID NO: 7]
QSISSY
[SEQ ID NO: 8]
AASS
[SEQ ID NO: 9]
QQSYSTPLTF
[SEQ ID NO: 10]
QVQLQESGPGLVKPSETLSLTCTVSGGSVSSGSYYWSWIRQPPGKGL
EWIGYIYYSGSTNYNPSLKSRVTISVDTSKNQFSLKLSSVTAADTAV
YYCAREGKNGAFDIWGQGTMVTVSS
[SEQ ID NO: 11]
RHQMTQSPSSLSASVGDRVTITCRASQSISSYLNWYQQKPGKAPKLL
IYAASSLQSGVPSRFSGSGSGTDFTLTISSLQPEDFATYYCQQSYST
PLTFGGGTKVEIKRT
[SEQ ID NO: 12]
CAGGTTCAGCTTCAGGAGAGTGGCCCAGGCCTGGTGAAGCCAAGTGA
GACTCTCAGCTTGACTTGCACAGTTTCTGGAGGCAGTGTCTCCTCAG
GCAGCTATTATTGGTCCTGGATTCGGCAGCCCCCTGGGAAAGGCCTG
GAGTGGATTGGGTACATATATTACAGTGGCAGCACAAATTACAATCC
ATCCCTGAAGTCTCGAGTAACTATCAGTGTGGACACAAGCAAGAATC
AGTTTTCACTCAAACTGTCTTCTGTGACTGCTGCTGACACTGCTGTT
TATTATTGTGCCAGGGAGGGGAAAAATGGGGCATTTGATATTTGGGG
TCAGGGCACAATGGTGACAGTCAGCTCT
[SEQ ID NO: 13]
CGCCATCAGATGACTCAGTCCCCCTCCAGTCTTTCTGCCTCAGTTGG
GGATAGAGTGACCATCACATGCAGAGCAAGTCAGAGCATATCATCCT
ATCTGAACTGGTACCAGCAGAAGCCAGGGAAAGCCCCCAAATTGCTG
ATTTATGCAGCCTCAAGTCTCCAGAGTGGGGTGCCAAGCAGGTTCTC
AGGCAGTGGCAGTGGGACAGATTTCACATTGACAATCAGCTCCCTCC
AACCTGAAGATTTTGCCACCTACTATTGCCAGCAATCCTACAGCACG
CCCCTGACTTTTGGAGGTGGCACAAAGGTAGAGATCAAGAGGACT
[SEQ ID NO: 14]
QVQLQESGPGLVKPSETLSLTCTVSGGSVSSGSYYWSWIRQPPGKGL
EWIGYIYYSGSTNYNPSLKSRVTISVDTSKNQFSLKLSSVTAADTAV
YYCAREGKNGAFDIWGQGTMVTVSSGGGGSGGGGSGGGGSRHQMTQS
PSSLSASVGDRVTITCRASQSISSYLNWYQQKPGKAPKLLIYAASSL
QSGVPSRFSGSGSGTDFTLTISSLQPEDFATYYCQQSYSTPLTFGGG
TKVEIKRT
[SEQ ID NO: 15]
CAGGTTCAGCTTCAGGAGAGTGGCCCAGGCCTGGTGAAGCCAAGTGA
GACTCTCAGCTTGACTTGCACAGTTTCTGGAGGCAGTGTCTCCTCAG
GCAGCTATTATTGGTCCTGGATTCGGCAGCCCCCTGGGAAAGGCCTG
GAGTGGATTGGGTACATATATTACAGTGGCAGCACAAATTACAATCC
ATCCCTGAAGTCTCGAGTAACTATCAGTGTGGACACAAGCAAGAATC
AGTTTTCACTCAAACTGTCTTCTGTGACTGCTGCTGACACTGCTGTT
TATTATTGTGCCAGGGAGGGGAAAAATGGGGCATTTGATATTTGGGG
TCAGGGCACAATGGTGACAGTCAGCTCTGGAGGTGGAGGCTCAGGAG
GAGGAGGCAGTGGAGGTGGTGGGTCACGCCATCAGATGACTCAGTCC
CCCTCCAGTCTTTCTGCCTCAGTTGGGGATAGAGTGACCATCACATG
CAGAGCAAGTCAGAGCATATCATCCTATCTGAACTGGTACCAGCAGA
AGCCAGGGAAAGCCCCCAAATTGCTGATTTATGCAGCCTCAAGTCTC
CAGAGTGGGGTGCCAAGCAGGTTCTCAGGCAGTGGCAGTGGGACAGA
TTTCACATTGACAATCAGCTCCCTCCAACCTGAAGATTTTGCCACCT
ACTATTGCCAGCAATCCTACAGCACGCCCCTGACTTTTGGAGGTGGC
ACAAAGGTAGAGATCAAGAGGACT

As used herein, the term “a conservative sequence modification” refers to an amino acid modification that does not significantly affect or alter the binding characteristics of the presently disclosed mesothelin-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 10 or SEQ ID NO: 11) may contain substitutions (e.g., conservative substitutions), insertions, or deletions relative to the specified sequence(s), but retain the ability to bind to a target antigen (e.g., mesothelin). In certain embodiments, a total of 1 to 10 amino acids are substituted, inserted and/or deleted in a specific sequence (e.g., SEQ ID NO: 10 or SEQ ID NO: 11). In certain embodiments, substitutions, insertions, or deletions occur in regions outside the CDRs (e.g., in the FRs) of the extracellular antigen-binding domain. In certain embodiments, the extracellular antigen-binding domain comprises a V_{H} comprising the amino acid sequence set forth in SEQ ID NO: 10 and a V_{L} comprising the amino acid sequence set forth in SEQ ID NO: 11, including post-translational modifications of these sequences (SEQ ID NO: 10 and SEQ ID NO: 11).

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

5.4.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8, CD28, CD3ζ, CD4, 4-1BB, OX40, ICOS, CD84, CD166, CD8a, CD8b, ICAM-1, CTLA-4, CD27, CD40, NKGD2, a synthetic peptide (not based on a protein associated with the immune response), or a combination thereof.

In certain embodiments, the transmembrane domain of the CAR comprises a CD8 polypeptide (e.g., a transmembrane domain of CD8).

In certain embodiments, the CD8 polypeptide comprises or consists of an amino acid sequence that is at least about 85%, about 90%, about 95%, about 96%, about 97%, about 98%, about 99% or about 100% homologous or identical to the sequence having a NCBI Reference No: NP_001139345.1 (SEQ ID NO: 19) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 19, which is at least 20, or at least 30, or at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 19. In certain embodiments, the transmembrane domain of the CAR comprises a CD8 polypeptide comprising or having amino acids 137 to 209 of SEQ ID NO: 19.

[SEQ ID NO: 19]
MALPVTALLLPLALLLHAARPSQFRVSPLDRTWNLGETVELKCQVLLS
NPTSGCSWLFQPRGAAASPTFLLYLSQNKPKAAEGLDTQRFSGKRLGD
TFVLTLSDFRRENEGYYFCSALSNSIMYFSHFVPVFLPAKPTTTPAPR
PPTPAPTIASQPLSLRPEACRPAAGGAVHTRGLDFACDIYIWAPLAGT
CGVLLLSLVITLYCNHRNRRRVCKCPRPVVKSGDKPSLSARYV

In certain embodiments, the CD8 polypeptide comprises or consists of an amino acid sequence that is at least about 85%, about 90%, about 95%, about 96%, about 97%, about 98%, about 99% or about 100% homologous or identical to the sequence having a NCBI Reference No: AAA92533.1 (SEQ ID NO: 20) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 20, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 20. In certain embodiments, the transmembrane domain of the CAR comprises a CD8 polypeptide comprising or having amino acids 151 to 219 of SEQ ID NO: 20.

| [SEQ ID NO: 20] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the transmembrane domain of the CAR comprises a CD8 polypeptide comprising or having the amino acid sequence set forth in SEQ ID NO: 21, which is provided below:

[SEQ ID NO: 21]
STTTKPVLRTPSPVHPTGTSQPQRPEDCRPRGSVKGTGLDFACDIYIW
APLAGICVALLLSLIITLICY

In accordance with the presently disclosed subject matter, a “CD8 nucleic acid molecule” refers to a polynucleotide encoding a CD8 polypeptide.

In certain embodiments, an exemplary CD8 nucleic acid molecule encoding the CD8 polypeptide consisting of the amino acid sequence set forth in SEQ ID NO: 21 is set forth in SEQ ID NO: 22, which is provided below.

[SEQ ID NO: 22]
TCTACTACTACCAAGCCAGTGCTGCGAACTCCCTCACCTGTGCACCCTA
CCGGGACATCTCAGCCCCAGAGACCAGAAGATTGTCGGCCCCGTGGCTC
AGTGAAGGGGACCGGATTGGACTTCGCCTGTGATATTTACATCTGGGCA
CCCTTGGCCGGAATCTGCGTGGCCCTTCTGCTGTCCTTGATCATCACTC
TCATCTGCTAC

In certain embodiments, the transmembrane domain of a presently disclosed CAR comprises a CD28 polypeptide (e.g., a transmembrane domain of CD28).

The CD28 polypeptide can consist of an amino acid sequence that is at least about 85%, about 90%, about 95%, about 96%, about 97%, about 98%, about 99% or 100% homologous or identical to the sequence having a NCBI Reference No: P10747 or NP_006130 (SEQ ID No: 2), or fragments thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 23 which is at least 20, or at least 30, or at least 40, or at least 50, and up to 220 amino acids in length. In non-limiting various embodiments, the CD28 polypeptide comprises or consists of an amino acid sequence of amino acids 1 to 220, 1 to 50, 50 to 100, 100 to 150, 150 to 200, 153 to 179. or 200 to 220 of SEQ ID NO: 23. In certain embodiments, the transmembrane domain of the CAR comprises a CD28 polypeptide comprising or consisting of amino acids 153 to 179 of SEQ ID NO: 23. SEQ ID NO: 23 is provided below:

| [SEQ ID NO: 23] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide.

In certain embodiments, an exemplar CD28 nucleic acid molecule encoding the CD28 polypeptide consisting of amino acids 153 to 179 of SEQ ID NO: 23 is set forth in SEQ ID NO: 24, which is provided below.

[SEQ ID NO: 24]
ttttgggtgctggtggtggttggtggagtcctggcttgctatagcttg
ctagtaacagtggcctttattattttctgggtg

In certain embodiments, the transmembrane domain of the CAR comprises a CD28 polypeptide comprises or consists of the amino acid sequence set forth in SEQ ID NO: 25. SEQ ID NO: 25 is provided below:

[SEQ ID NO: 25]
FWVLVVVGGV LACYSLLVTV AFIIFWV

An exemplary nucleic acid sequence encoding the amino acid sequence of SEQ ID NO: 25 is set forth in SEQ ID NO: 26, which is provided below.

[SEQ ID NO: 26]
TTTTGGGTGCTGGTGGTGGTTGGTGGAGTCCTGGCTTGCTATAGCTTG
CTAGTAACAGTGGCCTTTATTATTTTCTGGGTG

In certain non-limiting embodiments, a CAR further comprises a spacer region that links the extracellular antigen-binding domain to the transmembrane domain. The spacer region can be flexible enough to allow the antigen binding domain to orient in different directions to facilitate antigen recognition.

In certain non-limiting embodiments, the hinge/spacer region of the CAR comprises a native or modified hinge region of CD8, CD28, CD3ζ, CD40, 4-1BB, OX40, CD84, CD166, CD8a, CD8b, ICOS, ICAM-1, CTLA-4, CD27, CD40, NKGD2, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 23), a portion of a CD8 polypeptide (e.g., a portion of SEQ ID NO: 19, or a portion of SEQ ID NO: 20), a variation of any of the foregoing which is at least about 80%, at least about 85%, at least about 90%, at least about 95%, or at least about 100% homologous or identical thereto, or a synthetic spacer sequence.

5.4.3.3. Intracellular Signaling Domain of a CAR

In certain non-limiting embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the sequence having a NCBI Reference No: NP_932170 (SEQ ID NO: 27), or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 27, which is at least 20, or at least 30, or at least 40, or at least 50, and up to 164 amino acids in length. Alternatively or additionally, in non-limiting various embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 27. In certain embodiments, the intracellular signaling domain of the CAR comprises a CD3ζ polypeptide comprising or having amino acids 52 to 164 of SEQ ID NO: 27. SEQ ID NO: 27 is provided below:

| [SEQ ID NO: 27] | |
|---|---|
| 1 | MKWKALFTAA ILQAQLPITE AQSFGLLDPK LCYLLDGILF IYGVILTALE LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a modified CD3ζ polypeptide. In certain embodiments, the modified CD3ζ polypeptide is one disclosed in International Patent Publication No. WO2019/133969, which is incorporated hereby in its entirety.

In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 28 or a fragment thereof, and/or may optionally comprise up to one or up to two or up to three conservative amino acid substitutions. SEQ ID NO: 28 is provided below:

[SEQ ID NO: 28]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleic acid sequence encoding the amino acid sequence of SEQ ID NO: 28 is set forth in SEQ ID NO: 29, which is provided below.

[SEQ ID NO: 29]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtTcaatgaactgcagaaagataagat
ggcggaggcctTcagtgagattgggatgaaaggcgagcgccggaggggca
aggggcacgatggcctttTccaggggctcagtacagccaccaaggacacc
tTcgacgcccttcacatgcaggccctgccccctcgc

In certain non-limiting embodiments, the intracellular signaling domain of the CAR further comprises at least a co-stimulatory signaling region. In certain embodiments, the co-stimulatory region comprises at least one co-stimulatory molecule or a portion thereof. In certain embodiments, the co-stimulatory signaling region comprises an intracellular domain of at least one co-stimulatory molecule or a portion thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 23), or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 23 which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 23. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 23.

An exemplary nucleic acid sequence encoding amino acids 180 to 220 of SEQ ID NO: 23 is set forth in SEQ ID NO: 30, which is provided below.

[SEQ ID NO: 30]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_031668.3 (SEQ ID NO: 31), or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 31 which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 114 to 220, 150 to 200, 178 to 218, or 200 to 220 of SEQ ID NO: 31. In certain embodiments, the co-stimulatory signaling region of a presently disclosed CAR comprises a CD28 polypeptide that comprises or consists of the amino acids 178 to 218 of SEQ ID NO: 31. SEQ ID NO: 31 is provided below:

| [SEQ ID NO: 31] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

An exemplary nucleotide sequence encoding amino acids 178 to 218 of SEQ ID NO: 31 is set forth in SEQ ID NO: 32, which is provided below.

[SEQ ID NO: 32]
aat agtagaagga acagactcct tcaaagtgac tacatgaaca
tgactccccg gaggcctggg ctcactcgaa agccttacca
gccctacgcc cctgccagag actttgcagc gtaccgcccc

In certain embodiments, the intracellular signaling domain of the CAR comprises a co-stimulatory signaling region that comprises two co-stimulatory molecules or portions thereof (e.g., intracellular domains of the co-stimulatory molecules), e.g., intracellular domains of CD28 and 4-1BB, or intracellular domains of CD28 and OX40.

In certain non-limiting embodiments, the intracellular signaling domain of the CAR does not comprise a co-stimulatory signaling region, i.e., the CAR is a first-generation CAR. For example, the intracellular signaling domain of the CAR does not comprise an intracellular signaling domain of a co-stimulatory molecule, e.g., 4-1BB, CD28, etc. The co-stimulatory signaling domain comprised in the CAR may result in uncontrolled proliferation. The activation of c-Kit comprised in the cells can be controlled. For example, as disclosed in Section 5.2, the c-Kit mutant can be operably linked to a promoter, e.g., an inducible promoter, which can control the activation of c-Kit, e.g., with the control of the inducible promoter, c-Kit is activated only upon the activation of the cells comprising the c-Kit mutant (e.g., T cells or CAR-T cells).

5.4.3.4. Exemplified CARs

In certain embodiments, the CAR is a mesothelin-targeted CAR. In certain embodiments, the CAR comprises (a) an extracellular antigen-binding domain comprising a V_{H} CDR1 having the amino acid sequence set forth in SEQ ID NO: 4, a V_{H} CDR2 having the amino acid sequence set forth in SEQ ID NO: 5, a V_{H} CDR3 having the amino acid sequence set forth in SEQ ID NO: 6, a V_{L} CDR1 having the amino acid sequence set forth in SEQ ID NO: 7, a V_{L} CDR2 having the amino acid sequence set forth in SEQ ID NO: 8, and a V_{L} CDR3 having the amino acid sequence set forth in SEQ ID NO: 9; (b) a transmembrane domain comprising a transmembrane domain of CD28, and (c) an intracellular signaling domain comprising (i) a CD3ζ polypeptide, and (ii) a co-stimulatory signaling region comprising a CD28 polypeptide (e.g., an intracellular domain of CD28).

In certain embodiments, the CAR is a mesothelin-targeted CAR. In certain embodiments, the CAR comprises (a) an extracellular antigen-binding domain comprising a V_{H} CDR1 consisting of the amino acid sequence set forth in SEQ ID NO: 4, a V_{H} CDR2 consisting of the amino acid sequence set forth in SEQ ID NO: 5, a V_{H} CDR3 consisting of the amino acid sequence set forth in SEQ ID NO: 6, a V_{L} CDR1 consisting of the amino acid sequence set forth in SEQ ID NO: 7, a V_{L} CDR2 consisting of the amino acid sequence set forth in SEQ ID NO: 8, and a V_{L} CDR3 consisting of the amino acid sequence set forth in SEQ ID NO: 9; (b) a transmembrane domain comprising a transmembrane domain of CD8, and (c) an intracellular signaling domain comprising a CD3ζ polypeptide, and does not comprise a co-stimulatory signaling region.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

5.4.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

The presently disclosed subject matter provides polypeptide compositions comprising a mesothelin-targeted chimeric antigen receptor (CAR) and a dominant negative form of programmed death 1 (PD-1 DN).

### 5.5. Dominant Negative Form of Programmed Death 1 (PD-1 DN)

In certain embodiments, the cells of the presently disclosed subject matter further comprise a dominant negative form of programmed death 1 (referred to as “PD-1 DN”).

The PD-1 DN can enhance the therapeutic efficacy of an immunoresponsive cell comprising a CAR. In certain embodiments, the PD-1 DN comprises (a) at least a portion of an extracellular domain of programmed death 1 (PD-1) comprising a ligand binding region, and (b) a transmembrane domain.

In certain embodiments, a cell, such as a T cell, is engineered to express a dominant negative form (DN form) of PD-1.

Malignant cells adapt to generate an immunosuppressive microenvironment that protects the cells from immune recognition and elimination (Sharpe et al., Dis. Model Mech. 8:337-350 (2015)). The immunosuppressive microenvironment puts limitations on immunotherapy methods. Details of DN forms of inhibitors of a cell-mediated immune response are disclosed in WO2017/040945 and WO2017/100428, the contents of each of which are incorporated herein in their entireties.

Programmed cell death protein 1 (PD-1) is a negative immune regulator of activated T cells upon engagement with its corresponding ligands, PD-L1 and PD-L2, expressed on endogenous macrophages and dendritic cells. PD-1 is a type I membrane protein of 268 amino acids. PD-1 consists of two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif. PD-1 negatively regulates TCR signals. SHP-1 and SHP-2 phosphatases bind to the cytoplasmic tail of PD-1 upon ligand binding. Upregulation of PD-L1 is one mechanism tumor cells use to evade the host immune system. In pre-clinical and clinical trials, PD-1 blockade by antagonistic antibodies induced anti-tumor responses mediated through the host endogenous immune system.

In certain embodiments, a PD-1 polypeptide consists of the amino acid with a GenBank No. NP_005009.2 (SEQ ID NO: 33), or fragments thereof. In certain embodiments, amino acids 1 to 20 of SEQ ID NO: 33 is the signal peptide (or peptide signal) of PD-1. In certain embodiments, amino acids 21 to 170 of SEQ ID NO: 33 is the extracellular domain of PD-1. In certain embodiments, amino acids 171 to 191 of SEQ ID NO: 33 is the transmembrane domain of PD-1. In certain embodiments, amino acids 192 to 288 of SEQ ID NO: 33 is the intracellular domain of PD-1. SEQ ID NO: 33 is provided below:

[SEQ ID NO: 33]
MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

In certain embodiments, the extracellular domain of PD-1 comprises a ligand binding domain (referred to as “extracellular ligand binding domain”). In certain embodiments, the extracellular ligand binding domain of PD-1 is fused to one or more heterologous polypeptide sequences, that is, the PD-1 DN is a chimeric sequence. For example, the extracellular ligand binding domain of PD-1 can be fused at its N-terminus to a signal peptide that is optionally a heterologous signal peptide, including various signal peptides described herein. In addition, the PD-1 DN can comprise a transmembrane domain that is optionally a heterologous transmembrane domain, including any of various transmembrane domains described herein.

In certain embodiments, the PD-1 DN comprises the extracellular domain of a PD-1 polypeptide (e.g., amino acids 21 to 170 of SEQ ID NO: 33) or a ligand binding portion thereof (e.g., amino acids 21 to 165 of SEQ ID NO: 33). A cell expressing such a PD-1 DN may lack the ability or have reduced ability to signal in a PD-1 immune checkpoint pathway. In certain embodiments, the PD-1 DN is a deletion mutant having a deletion of the intracellular domain (e.g., the PD-1 DN lacks amino acids 192 to 288 of SEQ ID NO: 33) or a portion thereof. A PD-1 having a deletion of the intracellular domain may have reduced or inhibited immune checkpoint pathway mediated by PD-1. In certain embodiments, the PD-1 DN comprises the extracellular ligand binding domain of PD-1. In certain embodiments, the PD-1 DN comprises the extracellular ligand binding domain of a PD-1 polypeptide, and the transmembrane domain of a PD-1 polypeptide. In certain embodiments, the PD-1 DN comprises or consists of amino acids 21 to 165 of SEQ ID NO: 33.

An exemplary nucleotide sequence encoding amino acids 21 to 165 of SEQ ID NO: 33 is set forth in SEQ ID NO: 34, which is provided below.

[SEQ ID NO: 34]
CCAGGATGGTTCTTAGACTCCCCAGACAGGCCCTGGAACCCCCCCACCTT
CTCCCCAGCCCTGCTCGTGGTGACCGAAGGGGACAACGCCACCTTCACCT
GCAGCTTCTCCAACACATCGGAGAGCTTCGTGCTAAACTGGTACCGCATG
AGCCCCAGCAACCAGACGGACAAGCTGGCCGCTTTCCCCGAGGACCGCAG
CCAGCCCGGCCAGGACTGCCGCTTCCGTGTCACACAACTGCCCAACGGGC
GTGACTTCCACATGAGCGTGGTCAGGGCCCGGCGCAATGACAGCGGCACC
TACCTCTGTGGGGCCATCTCCCTGGCCCCCAAGGCGCAGATCAAAGAGAG
CCTGCGGGCAGAGCTCAGGGTGACAGAGAGAAGGGCAGAAGTGCCCACAG
CCCACCCCAGCCCCTCACCCAGGCCAGCCGGCCAG

In certain embodiments, the PD-1 DN further comprises a signal peptide, e.g., the PD-1 DN comprises the extracellular ligand binding domain of a PD-1 polypeptide, the transmembrane domain of a PD-1 polypeptide, and the signal peptide of a PD-1 polypeptide. In certain embodiments, the signal peptide comprises or consists of amino acids 1-20 of SEQ ID NO: 33. An exemplary nucleotide sequence encoding amino acids 1-20 of SEQ ID NO: 33 is set forth in SEQ ID NO: 35, which is provided below.

[SEQ ID NO: 35]
ATGCAGATCCCACAGGCGCCCTGGCCAGTCGTCTGGGCGGTGCTACAACT
GGGCTGGCGG

In certain embodiments, the PD-1 DN comprises or consists of amino acids 1 to 165 of SEQ ID NO: 33.

An exemplary nucleotide sequence encoding amino acids 1-165 of SEQ ID NO: 33 is set forth in SEQ ID NO: 36, which is provided below.

[SEQ ID NO: 36]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAG

In certain embodiments, the PD-1 DN comprises or consists of amino acids 21 to 151 of SEQ ID NO: 33. In certain embodiments, a PD-1 DN comprises or consists of amino acids 1 to 151 of SEQ ID NO: 33. In certain embodiments, a PD-1 DN comprises or consists of amino acids 21 to 151 of SEQ ID NO: 33. In certain embodiments, the PD-1 DN comprises or consists of an amino acid sequence starting at amino acid 21 of SEQ ID NO: 33 through an amino acid between amino acids 151 to 165 of SEQ ID NO: 33.

In certain embodiments, the PD-1 DN further comprises a CD8 polypeptide. In certain embodiments, the PD-1 DN comprises the extracellular domain of PD-1 or a portion thereof (e.g., the extracellular ligand binding domain) fused to the transmembrane domain and/or the hinge domain of CD8. In certain embodiments, the PD-1 DN comprises the transmembrane domain of CD8 (e.g., amino acids 183 to 203 of SEQ ID NO: 19). Such embodiments are representative of a chimeric DN form comprising a transmembrane domain from a different (heterologous) polypeptide. As described above, a PD-1 DN comprising a heterologous domain such as a transmembrane domain can optionally include additional sequence from the heterologous polypeptide. In certain embodiments, the PD-1 DN comprises an additional sequence from the heterologous polypeptide N-terminal of the transmembrane domain. In certain embodiments, the PD-1 DN comprises the hinge domain of CD8. In certain embodiments, the heterologous sequence comprises an additional N-terminal sequence of a CD8 polypeptide (e.g., amino acids 137 to 182 (or optionally starting at amino acids 138 or 139) of SEQ ID NO: 19). In certain embodiments, the PD-1 DN comprises an additional sequence from the heterologous polypeptide C-terminal of the transmembrane domain of CD8. In certain embodiments, the additional C-terminal sequence is amino acids 204 to 209 of SEQ ID NO: 19.

In certain embodiments, the PD-1 DN comprises the transmembrane domain of a CD8 polypeptide (e.g., amino acids 183 to 203 of SEQ ID NO: 19), a hinge domain of a CD8 polypeptide (e.g., amino acids 137 to 182 of SEQ ID NO: 19), and an additional C-terminal sequence of a CD8 polypeptide (e.g., amino acids 204 to 207 of SEQ ID NO: 19. In certain embodiments, the PD-1 DN comprises a CD8 polypeptide consisting of amino acids 137 to 207 of SEQ ID NO: 19.

An exemplary nucleotide sequence encoding amino acids 137-207 of SEQ ID NO: 19 is set forth in SEQ ID NO: 37, which is provided below:

[SEQ ID NO: 37]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAAC

In certain embodiments, the PD-1 DN comprises the transmembrane domain of a CD8 polypeptide (e.g., amino acids 183 to 203 of SEQ ID NO: 19), a hinge domain of a CD8 polypeptide (e.g., amino acids 137 to 182 of SEQ ID NO: 19), and an additional C-terminal sequence of a CD8 polypeptide (e.g., amino acids 204 to 209 of SEQ ID NO: 19. In certain embodiments, the PD-1 DN comprises a CD8 polypeptide having amino acids 137 to 209 of SEQ ID NO: 19.

An exemplary nucleotide sequence encoding amino acids 137-209 of SEQ ID NO: 19 is set forth in SEQ ID NO: 38, which is provided below:

[SEQ ID NO: 38]
CCCACCACGACGCCAGCGCCGCGACCACCAACCCCGGCGCCCACGATCGC
GTCGCAGCCCCTGTCCCTGCGCCCAGAGGCGTGCCGGCCAGCGGCGGGGG
GCGCAGTGCACACGAGGGGGCTGGACTTCGCCTGTGATATCTACATCTGG
GCGCCCCTGGCCGGGACTTGTGGGGTCCTTCTCCTGTCACTGGTTATCAC
CCTTTACTGCAACCACAGG

In certain embodiments, the PD-1 DN comprises the amino acid sequence set forth in SEQ ID NO: 39, which is provided below.

[SEQ ID NO: 39]
MQIPQAPWPVVWAVLQLGWRPGWELDSPDRPWNPPTESPALLVVTEGDNA
TFTCSFSNTSESFVLNWYRMSPSNOTDKLAAFPEDRSQPGQDCRFRVTQL
PNGRDFHMSVVRARRNDSGTYLCGAISLAPKAQIKESLRAELRVTERRAE
VPTAHPSPSPRPAGQAAAPTTTPAPRPPTPAPTIASQPLSLRPEACRPAA
GGAVHTRGLDFACDIYIWAPLAGTCGVLLLSLVITLYCN

An exemplary nucleotide sequence encoding the amino acid sequence set forth in SEQ ID NO: 39 is set forth in SEQ ID NO: 40, which is provided below:

[SEQ ID NO: 40]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGGCGGC
CGCACCCACCACGACGCCAGCGCCGCGACCACCAACCCCGGCGCCCACGA
TCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACAT
CTGGGCGCCCCTGGCCGGGACTTGTGGGGTCCTTCTCCTGTCACTGGTTA
TCACCCTTTACTGCAAC

In certain embodiments, the PD-1 DN comprises the amino acid sequence set forth in SEQ ID NO: 41, which is provided below.

[SEQ ID NO: 41]
MQIPQAPWPVVWAVLQLGWRPGWELDSPDRPWNPPTESPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQAAAPTTTPAPRPPTPAPTIASQPLSLRPEACRPAA
GGAVHTRGLDFACDIYIWAPLAGTCGVLLLSLVITLYCNHR.

An exemplary nucleotide sequence encoding the amino acid sequence set forth in SEQ ID NO: 41 is set forth in SEQ ID NO: 42, which is provided below:

[SEQ ID NO: 42]
ATGCAGATCCCACAGGCGCCCTGGCCAGTCGTCTGGGCGGTGCTACAACT
GGGCTGGCGGCCAGGATGGTTCTTAGACTCCCCAGACAGGCCCTGGAACC
CCCCCACCTTCTCCCCAGCCCTGCTCGTGGTGACCGAAGGGGACAACGCC
ACCTTCACCTGCAGCTTCTCCAACACATCGGAGAGCTTCGTGCTAAACTG
GTACCGCATGAGCCCCAGCAACCAGACGGACAAGCTGGCCGCTTTCCCCG
AGGACCGCAGCCAGCCCGGCCAGGACTGCCGCTTCCGTGTCACACAACTG
CCCAACGGGCGTGACTTCCACATGAGCGTGGTCAGGGCCCGGCGCAATGA
CAGCGGCACCTACCTCTGTGGGGCCATCTCCCTGGCCCCCAAGGCGCAGA
TCAAAGAGAGCCTGCGGGCAGAGCTCAGGGTGACAGAGAGAAGGGCAGAA
GTGCCCACAGCCCACCCCAGCCCCTCACCCAGGCCAGCCGGCCAGGCGGC
CGCACCCACCACGACGCCAGCGCCGCGACCACCAACCCCGGCGCCCACGA
TCGCGTCGCAGCCCCTGTCCCTGCGCCCAGAGGCGTGCCGGCCAGCGGCG
GGGGGCGCAGTGCACACGAGGGGGCTGGACTTCGCCTGTGATATCTACAT
CTGGGCGCCCCTGGCCGGGACTTGTGGGGTCCTTCTCCTGTCACTGGTTA
TCACCCTTTACTGCAACCACAGG

In certain non-limiting embodiments, the transmembrane domain of the PD-1 DN comprises a hydrophobic alpha helix that spans at least a portion of the membrane. Different transmembrane domains result in different receptor stability. In accordance with the presently disclosed subject matter, the transmembrane domain of the PD-1 DN can comprise a native or modified transmembrane domain of any polypeptide disclose herein, e.g., any transmembrane domain that can be comprised in a chimeric antigen receptor. In certain embodiments, the transmembrane domain is a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof. In certain embodiments, the transmembrane domain is a CD8 polypeptide. Detail of these transmembrane domains are described in Section 5.4.

### 5.6. Compositions and Vectors

The presently disclosed subject matter provides compositions comprising (a) a c-Kit mutant disclosed herein (e.g., disclosed in Section 5.2) and an antigen-recognizing receptor disclosed herein (e.g., disclosed in Section 5.4). Also provided are cells comprising such compositions.

In certain embodiments, the c-Kit mutant is operably linked to a first promoter. In certain embodiments, the antigen-recognizing receptor is operably linked to a second promoter.

Furthermore, the present discloses subject matter provides nuclei acid compositions comprising a first polynucleotide encoding a c-Kit mutant disclosed herein (e.g., disclosed in Section 5.2) and a second polynucleotide encoding an antigen-recognizing receptor disclosed herein (e.g., disclosed in Section 5.4). Also provided are cells comprising such nucleic acid compositions.

In certain embodiments, the nucleic acid composition further comprises a first promoter that is operably linked to the c-Kit mutant. In certain embodiments, the nucleic acid composition further comprises a second promoter that is operably linked to the antigen-recognizing receptor.

In certain embodiments, one or both of the first and second promoters are endogenous or exogenous. In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include an antigen-recognizing receptor (e.g., a CAR or a TCR), a retroviral vector is generally employed for transduction, however any other suitable viral vector or non-viral delivery system can be used. The antigen-recognizing receptor and the c-Kit mutant can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the c-Kit mutant and/or the antigen-recognizing receptor disclosed herein to a cell or a subject. In certain embodiments, a CRISPR system is used to deliver the c-Kit mutant and/or the antigen-recognizing receptor disclosed herein. In certain embodiments, zinc-finger nucleases are used to deliver the c-Kit mutant and/or the antigen-recognizing receptor disclosed herein. In certain embodiments, a TALEN system is used to deliver the c-Kit mutant and/or the antigen-recognizing receptor disclosed herein.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.7. Polypeptides and Analogs

Also included in the presently disclosed subject matter are a mesothelin, CD28, CD8, CD3ζ, and c-Kit polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, mesothelin, CD8, CD28, CD3ζ, and c-Kit). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., the c-Kit mutant). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 5.8. Administration

The presently disclosed subject matter also provides compositions comprising the presently disclosed cells.

Compositions comprising the presently disclosed cells can be provided systemically or directly to a subject for inducing and/or enhancing an immune response to an antigen and/or treating and/or preventing a neoplasm, a pathogen infection, or an infectious disease. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells or NK cells in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed cells and a pharmaceutically acceptable carrier. Administration can be autologous or heterologous. For example, cells can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a presently disclosed composition (e.g., a pharmaceutical composition comprising presently disclosed cells), it can be formulated in a unit dosage injectable form (solution, suspension, emulsion).

In certain embodiments, the composition further comprises an inhibitor of c-Kit (referred to as “c-Kit inhibitor”). The c-Kit inhibitor can inhibit the activity of c-Kit (e.g., kinase activity). In certain embodiments, the c-Kit inhibit specifically inhibits the c-Kit mutant, e.g., c-Kit D816V. In certain embodiments, the c-Kit inhibit is a multi-tyrosine kinase inhibitor. Non-liming examples of c-Kit inhibitors include Dasatinib, Midostaurin, ponatinib, imatinib.

### 5.9. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁴ and about 10⁶, between about 10⁵ and about 10⁹, or between about 10⁶ and about 10⁸ of the presently disclosed cells are administered to a human subject. More effective cells may be administered in even smaller numbers. In certain embodiments, at least about 1×10⁵, about 2×10⁵, about 3×10⁵, about 4×10⁵, or about 5×10⁵ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 5.10. Methods of Treatment

The cells and compositions comprising thereof of the presently disclosed subject matter can be used for the treatment and/or prevention of a neoplasia, pathogen infection, infectious disease, inflammatory disease, or graft rejection. Such cells can be administered to a subject (e.g., a human subject) in need thereof for the treatment or prevention of a solid tumor (e.g. mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and/or cholangiocarcinoma). In certain embodiments, the cell is a T cell. The T cell can be a CD4⁺ T cell or a CD8⁺ T cell. In certain embodiments, the T cell is a CD4⁺ T cell.

In certain embodiments, a cell of the neoplasm or tumor has a high expression level of mesothelin (referred to as “a high-MSLN expressing cell”). In certain embodiments, a high-MSLN expressing cell is a cell expressing mesothelin at an expression level of about 50 folds or more, about 50 folds or more, about 60 folds or more, about 70 folds or more, about 80 folds or more, about 90 folds or more, about 100 folds or more, about 150 folds or more, about 200 folds or more, about 300 folds or more, about 400 folds or more, or about 500 folds or more as compared to the mesothelin expression level of a normal cell.

In certain embodiments, a cell of the neoplasm or tumor has a low expression level of mesothelin (referred to as “a low-MSLN expressing cell”). In certain embodiments, a low-MSLN expressing cell is a cell expressing mesothelin at an expression level of about 50 folds or less, about 40 folds or less, about 30 folds or less, about 20 folds or less, about 10 folds or less, about 5 folds or less, about 4 folds or less, about 3 folds or less, or about 2 folds or less as compared to the mesothelin expression level of a normal cell.

In certain embodiments, the solid tumor is a lung cancer.

In certain embodiments, the solid tumor is a mesothelioma. In certain embodiments, the mesothelioma cell is a high-MSLN expressing cell. In certain embodiments, the cells used in treating a high-MSLN expressing mesothelioma cell comprise a CAR that does not comprise a co-stimulatory signaling region (e.g., a first-generation CAR).

The presently disclosed subject matter provides methods for inducing and/or increasing an immune response in a subject in need thereof. The presently disclosed cells and compositions comprising thereof can be used in a therapy or medicament. The presently disclosed cells and compositions comprising thereof can be used for treating and/or preventing a neoplasia in a subject. The presently disclosed cells and compositions comprising thereof can be used for prolonging the survival of a subject suffering from a neoplasia. The presently disclosed cells and compositions comprising thereof can also be used for treating and/or preventing a pathogen infection or other infectious disease in a subject, such as an immunocompromised human subject. Such methods comprise administering the presently disclosed cells or a composition (e.g., a pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are pleurally administered to the subject in need. The presently disclosed subject matter provides various methods of using the cells (e.g., T cells) or compositions comprising thereof. For example, the presently disclosed subject matter provides methods of reducing tumor burden in a subject. In certain embodiments, the method of reducing tumor burden comprises administering the presently disclosed cells or a composition comprising thereof to the subject. The presently disclosed cell can reduce the number of tumor cells, reduce tumor size, and/or eradicate the tumor in the subject. The tumor can be a solid tumor. Non-limiting examples of solid tumor include mesothelioma, lung cancer, pancreatic cancer, ovarian cancer, breast cancer, colon cancer, pleural tumor, glioblastoma, esophageal cancer, gastric cancer, synovial sarcoma, thymic carcinoma, endometrial carcinoma, stomach cancer, and cholangiocarcinoma.

The presently disclosed subject matter also provides methods of increasing or lengthening survival of a subject having a neoplasm. In certain embodiments, the method of increasing or lengthening survival of a subject having neoplasm comprises administering an effective amount of the presently disclosed immunoresponsive cells or a composition comprising thereof to the subject. The method can reduce or eradicate tumor burden in the subject. Additionally, the presently disclosed subject matter provides methods for increasing an immune response in a subject, comprising administering the presently disclosed cell or a composition comprising thereof to the subject. The presently disclosed subject matter further provides methods for treating and/or preventing a neoplasia in a subject, comprising administering the presently disclosed cells or a composition comprising thereof to the subject.

As used herein, the term “neoplasm” refers to a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasms can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In one embodiment, the neoplasm is a solid tumor. The neoplasm can a primary tumor or primary cancer. In addition, the neoplasm can be in metastatic status.

Cancers whose growth may be inhibited using the immunoresponsive cells of the presently disclosed subject matter comprise cancers typically responsive to immunotherapy. Non-limiting examples of cancers for treatment include mesothelioma, lung cancer (e.g. non-small cell lung cancer), pancreatic cancer, ovarian cancer, breast cancer (e.g., metastatic breast cancer, metastatic triple-negative breast cancer), colon cancer, pleural tumor, glioblastoma, esophageal cancer, gastric cancer, synovial sarcoma, thymic carcinoma, endometrial carcinoma, stomach cancer, cholangiocarcinoma, cervical cancer, and salivary gland cancer. Additionally, the presently disclosed subject matter comprises refractory or recurrent malignancies whose growth may be inhibited using the immunoresponsive cells of the presently disclosed subject matter.

Examples of other neoplasms or cancers that may be treated using the methods of the presently disclosed subject matter include bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of a neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

As a consequence of surface expression of an antigen-recognizing receptor that binds to a tumor antigen and a c-Kit mutant that enhances the anti-tumor effect of the immunoresponsive cell, adoptively transferred T or NK cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed immunoresponsive cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CART cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

In addition, the presently disclosed subject matter provides methods of preventing and/or treating an inflammatory disease in a subject. In certain embodiments, the method comprises administering the presently disclosed cells or a composition comprising thereof to the subject. In certain embodiments, the cell is an immunoinhibitory cell. In certain embodiments, the immunoinhibitory cell is a regulatory T cell. In one embodiment, the inflammatory disease is pancreatitis. In certain embodiments, the subject is a human. In certain embodiments, the subject is a recipient of an organ transplant, e.g., a recipient of a pancreas transplant.

Furthermore, the presently disclosed subject matter provides methods of preventing graft rejection in a subject who is a recipient of an organ transplant. In certain embodiments, the method comprises administering the presently disclosed cells or a composition comprising thereof to the subject. In certain embodiments, the cell is an immunoinhibitory cell. In certain embodiments, the immunoinhibitory cell is a regulatory T cell. In certain embodiments the subject is a human. In a further embodiment, the subject is a recipient of a pancreas transplant.

In certain embodiments, the method disclosed herein further comprises administering to the subject an inhibitor of c-Kit (e.g., one disclosed in Section 5.8). The c-Kit inhibitor can inhibit the activity of c-Kit (e.g., kinase activity). In certain embodiments, the c-Kit inhibit specifically inhibits the c-Kit mutant, e.g., c-Kit D816V. In certain embodiments, the c-Kit inhibit is a multi-tyrosine kinase inhibitor. Non-liming examples of c-Kit inhibitors include Dasatinib, Midostaurin, ponatinib, imatinib.

### 5.11. Kits

The presently disclosed subject matter provides kits for inducing and/or enhancing an immune response and/or treating and/or preventing a neoplasia or a pathogen infection, or an immune disorder in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding a c-Kit mutant in expressible form, which may optionally be comprised in the same or different vectors.

If desired, the cells and/or nucleic acid molecules are provided together with instructions for administering the cells or nucleic acid molecules to a subject having or at risk of developing a neoplasia or pathogen or immune disorder. The instructions generally include information about the use of the composition for the treatment and/or prevention of neoplasia or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Generation of Constructs

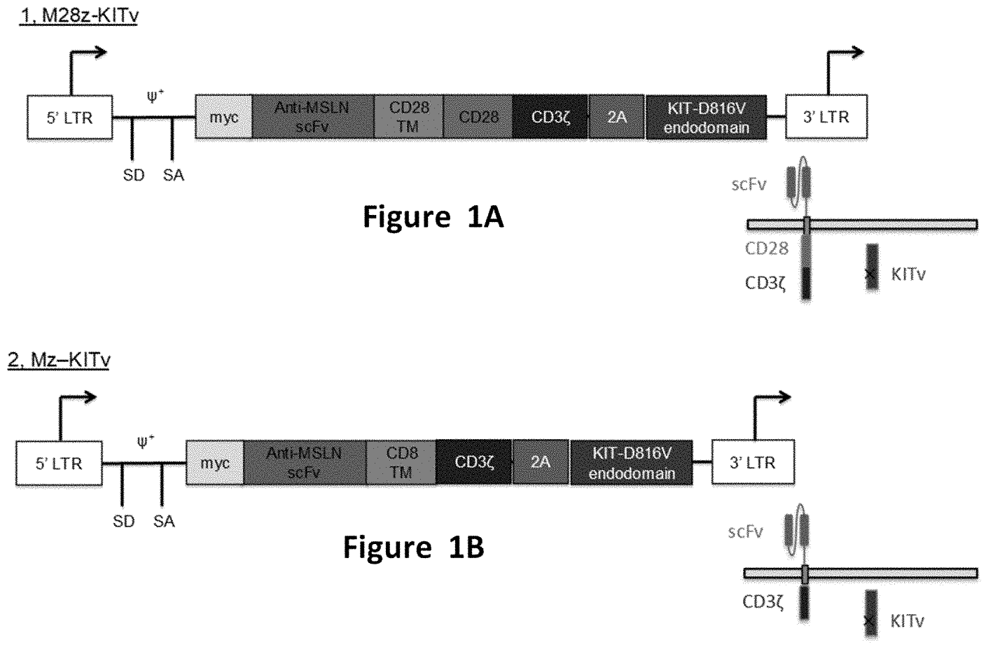

Two constructs of the presently disclose subject matter were generated. One construct is designated as “M28z-KITv” (also referred to as “M28z-KITm”). The structure of M28z-KITv is shown in FIG. 1A. As shown in FIG. 1A, M28z-KITv comprises a c-Kit mutant (e.g., c-Kit D816V) and a second generation CAR comprising an anti-MSKN scFv, a transmembrane domain comprising a CD28 polypeptide (e.g., a CD28 transmembrane domain or a portion thereof), an intracellular domain that comprises a CD3ζ and a CD28 polypeptide (e.g., an intracellular domain of CD28). Another construct is designed as “Mz-KITv”. The structure of Mz-KITv is shown in FIG. 1B. As shown in FIG. 1B, Mz-KITv comprises a c-Kit mutant (e.g., c-Kit D816V) and a first generation CAR comprising an anti-MSKN scFv, a transmembrane domain comprising a CD28 polypeptide (e.g., a CD28 transmembrane domain or a portion thereof), an intracellular domain that comprises a CD3ζ.

### Example 2—Transduction, Activation of pKIT signaling, Expansion, and Proliferation of the Constructs

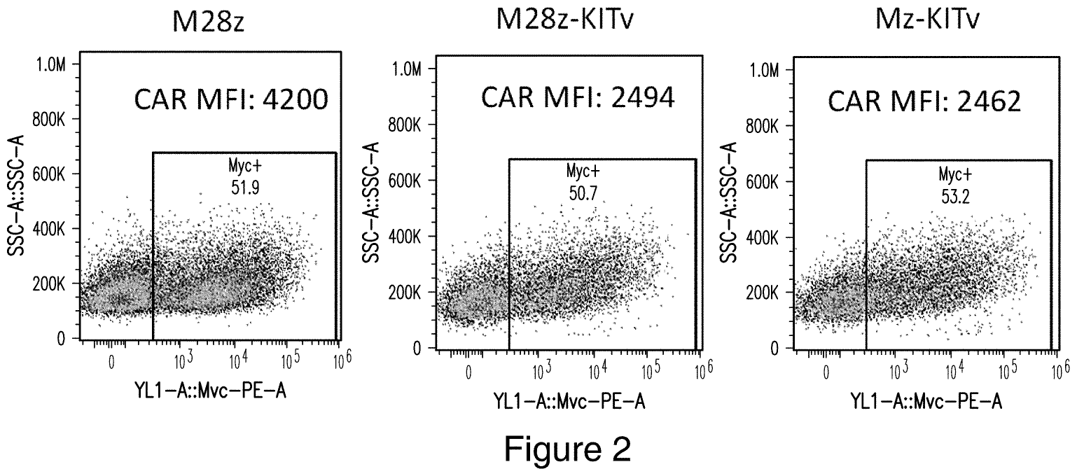

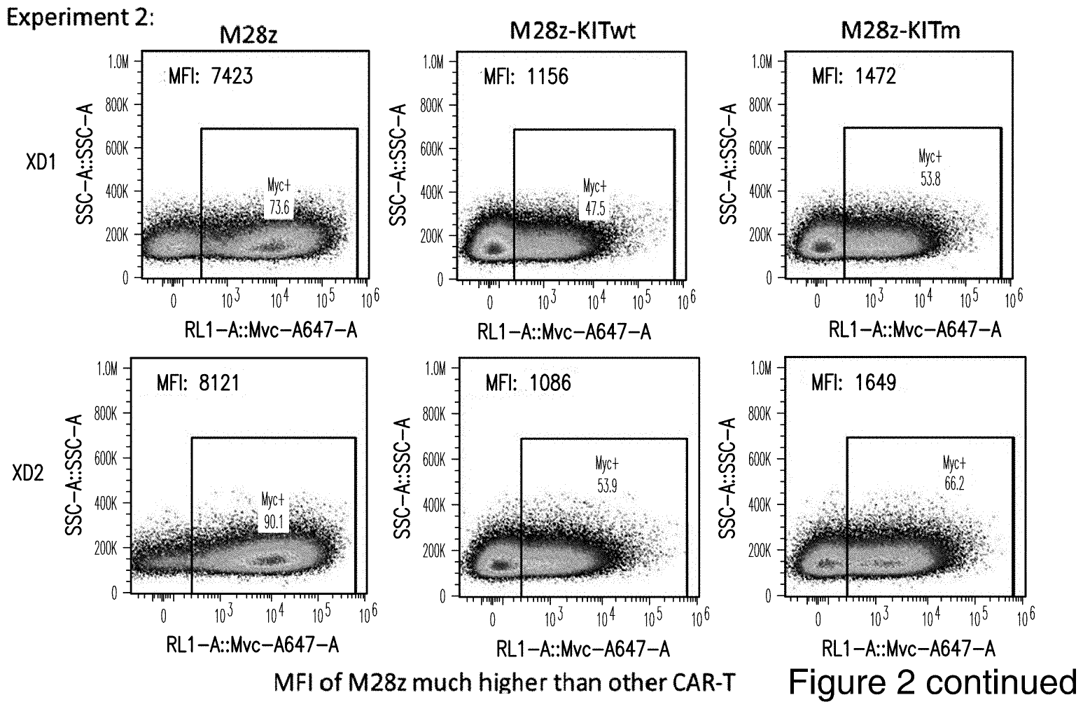

The transduction, activation of pKIT signaling, expansion, and proliferation of M28z-KITv and Mz-KITv were tested. M28z comprises the same CAR construct as M28z-KITv but does not comprise a Kit mutant. The transduction results are shown in FIG. 2. As shown in FIG. 2, both M28z-KITv and Mz-KITv had good transduction. MFI value for T cells comprising M28z was higher than T cells comprising M28z-KITv (referred to as “M28z-KITv CAR T cells”) or T cells comprising Mz-KITv (referred to as “Mz-KITv CAR T cells”).

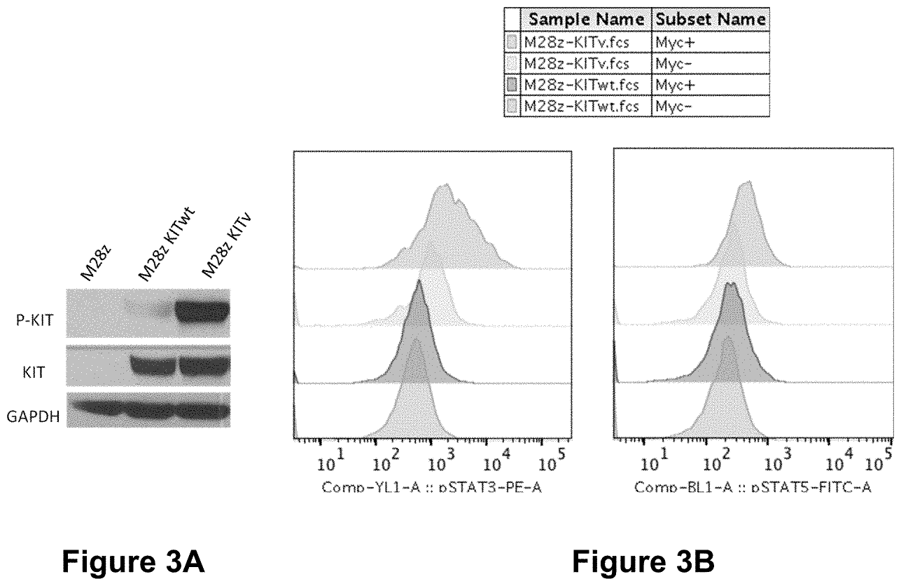

The results of the constructs for the ability to activate pKIT signaling are shown in FIGS. 3A and 3B. As shown in FIGS. 3A and 3B, M28z-KITv CAR T cells constitutively exhibited activated pKIT signaling. As shown in FIG. 3A, M28z-KITv CAR T cells exhibited pKIT activity without SCF, while M28z CAR T cells did not express KIT protein. As shown in FIG. 3B, M28z-KITv CAR T cells exhibited higher p-STAT3 and p-STAT5 activity than M28z-KITwt CAR T cells control.

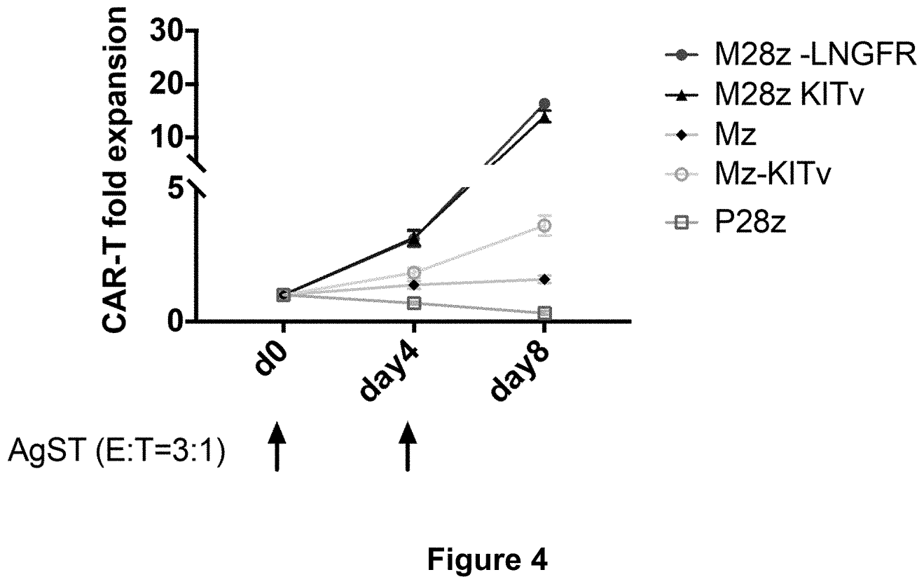

The CAR T cells expansion results are shown in FIG. 4. FIG. 4 shows cumulative expansion of CAR T cells during serial co-culture. T-cell were re-stimulated with A549GM tumor cells (E:T=3:1). As shown in FIG. 4, M28z-KITv CAR T cells and M28z CAR T cells exhibited similar expansion level, while Mz-KITv CAR T cells exhibited less expansion. A549GM was non-small cell lung cancer cell line with overexpression of GFP-luciferase and mesothelin.

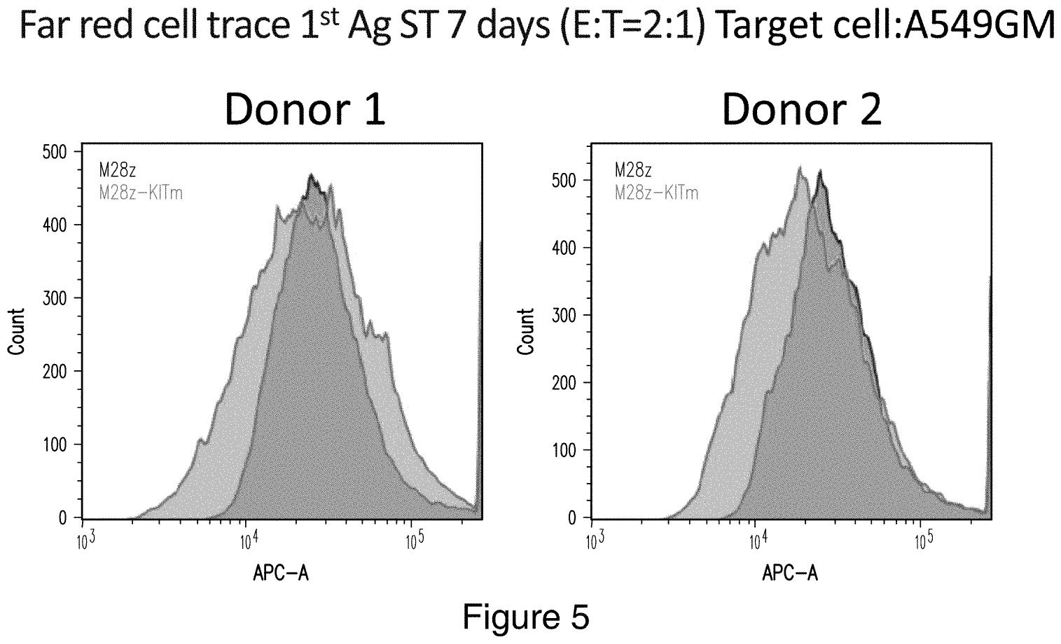

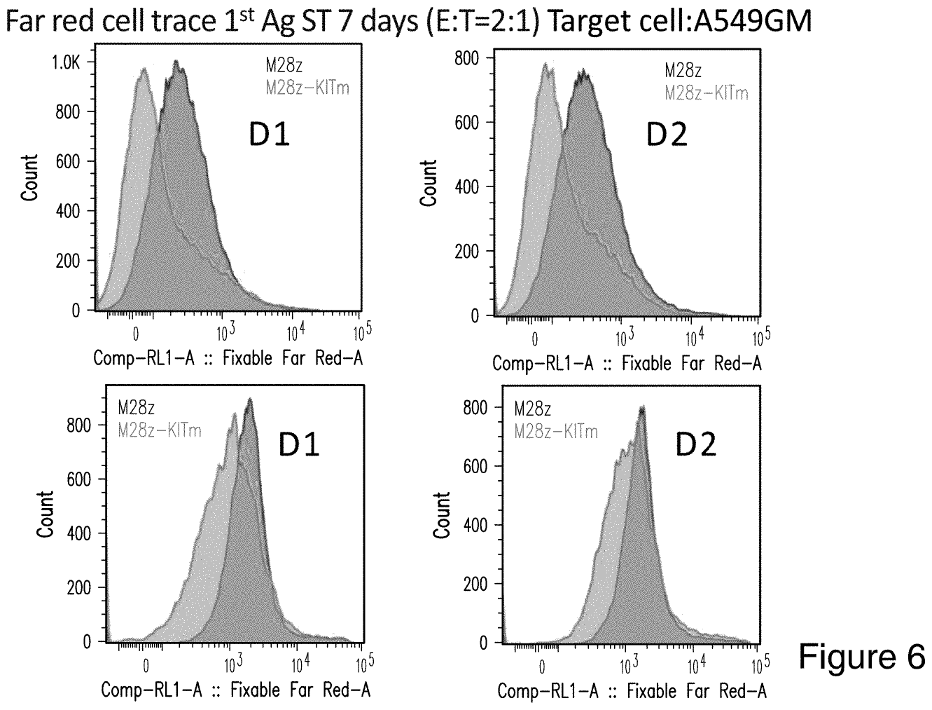

The proliferation results are shown in FIGS. 5 and 6. Far red cell trace staining of CAR T cells is measured after 7 days first antigen stimulation (E:T=2:1). The target cell was A549GM. As shown in FIGS. 5 and 6, M28z-KITm CAR T cells exhibited higher proliferation as compared to M28z CAR T cells.

Thus, the cKIT costimulatory constructs demonstrated successful transduction, and antigen-specific activation, and proliferation.

### Example 3—In Vitro Cytolytic Activity of the Presently Disclosed Constructs

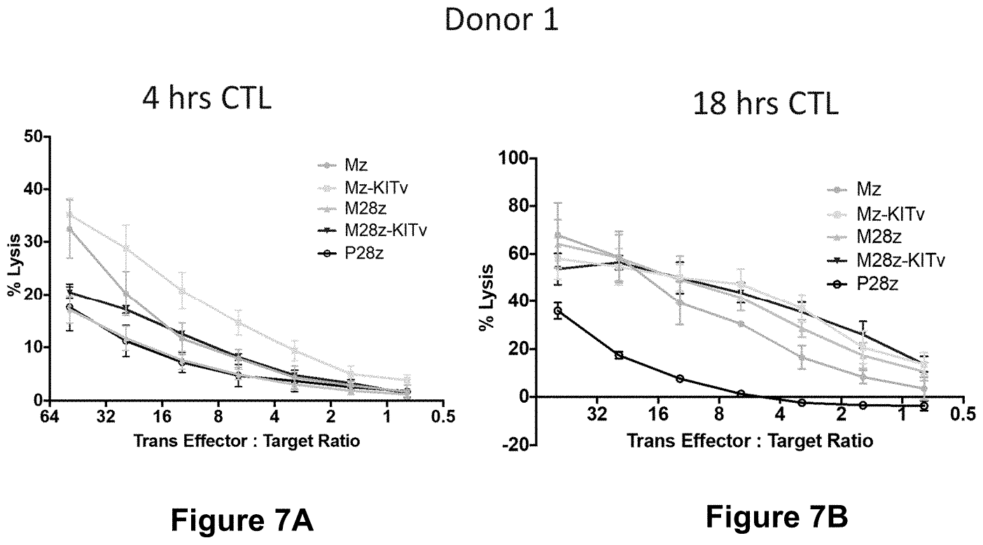

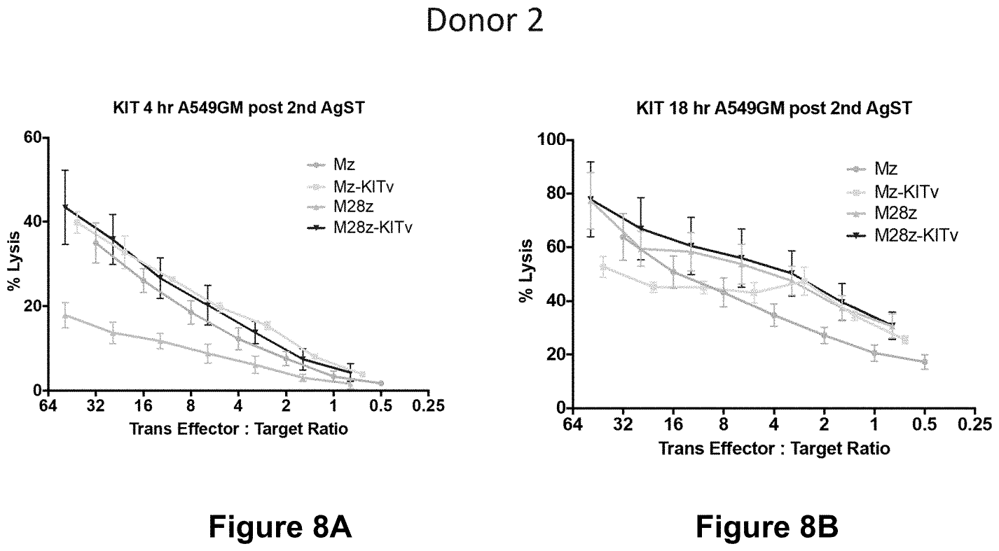

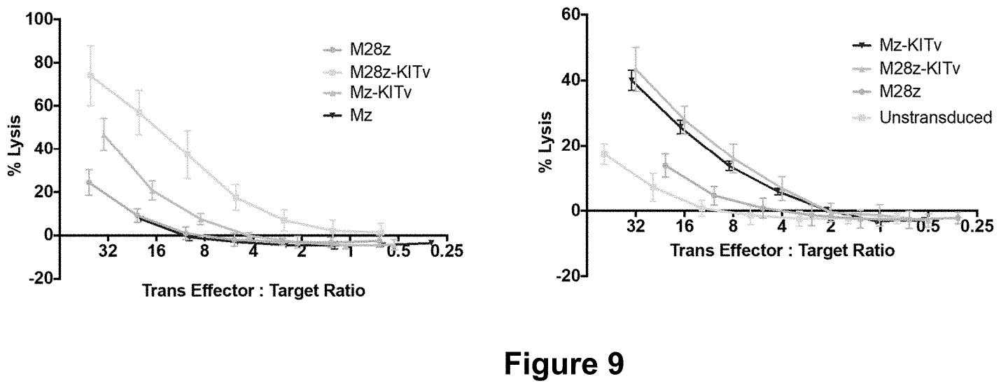

The in vitro cytolytic activity of M28z-KITv CAR T cells and Mz-KITv CAR T cells towards high MSLN expressing cells and low MSLN expressing cells were assessed. The results are shown in FIGS. 7A-7B, 8A-8B, and 9. As shown in FIGS. 7A-7B and 8A-8B, M28z-KITv CAR T cells and Mz-KITv CAR T cells killed high MSLN tumor cells faster than M28z CART cells and Mz CAR T cells, e.g., at 4 hours M28z-KITv CAR T cells killed more tumor cells than M28z CAR T cells, and Mz-KITv CAR T cells killed more tumor cells than Mz CAR T cells (see FIGS. 7A and 8A), while at 18 hours no significant difference was seen (see FIGS. 7B and 8B).

However, for low MSLN expressing tumor cells, i.e., A549G cells, M28z-KITv CART cells and Mz-KITv CART cells exhibited increased killing capability. At 18 hours, M28z-KITv CAR T cells and Mz-KITv CAR T cells killed more low MSLN A549G tumor cells than M28z CAR T cells.

### Example 4—PD1 Expression and T Cells Status of the Constructs after Antigen Stimulation

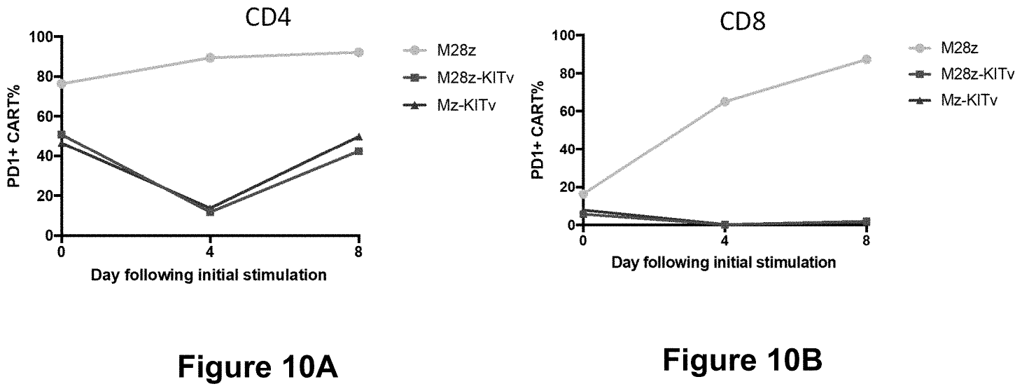

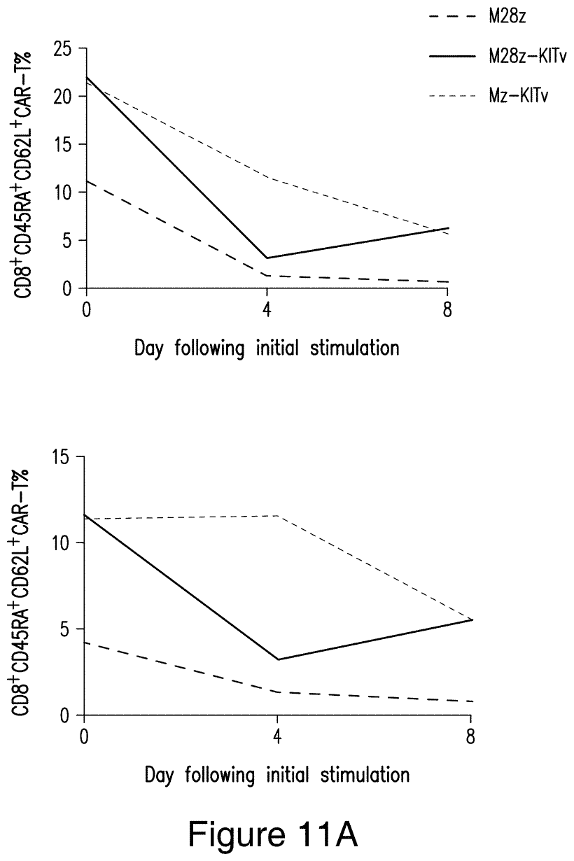

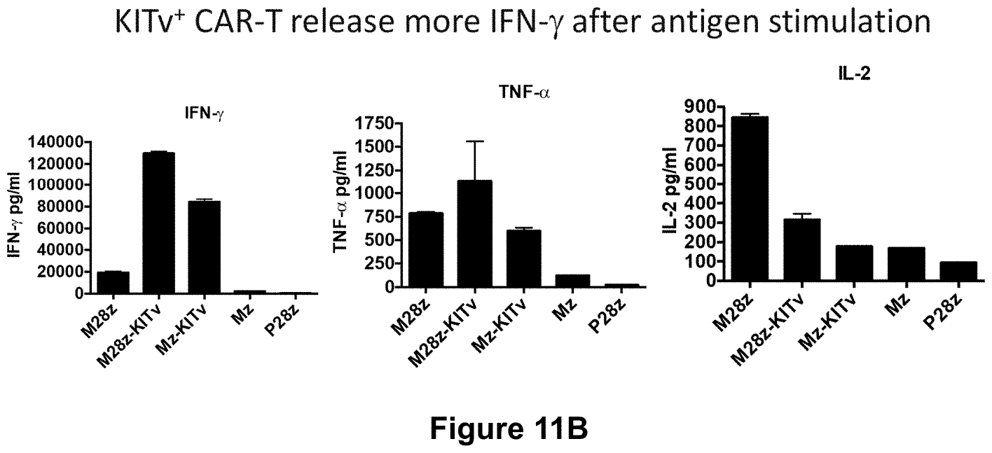

The PD1 expression after antigen stimulation was assessed. After stimulation with A549GM cells for every 4 days (E:T=3:1), FACS were measured. The results for PD1 expression are shown in FIGS. 10A and 10B. As shown in FIGS. 10A and 10B, M28z-KITv CAR T cells and Mz-KITv CAR T cells showed less PD1+ expression after antigen stimulation in both CD4⁺ T cells (see FIG. 10A) and CD8⁺ T cells (see FIG. 10B) as compared to M28z CART cells. The results for the T cells status are shown in FIG. 11A. As shown in FIG. 11A, M28z-KITv CAR T cells and Mz-KITv CAR T cells showed more stem cell-like memory T cells (T_{SCM} cells) after antigen stimulation. T_{SCM} cells have higher potential to kill tumor cells. M28z-KITv CAR T cells and Mz-KITv CAR T cells secreted higher IFN-γ and TNF-α than M28z and Mz CAR T cells, albeit less IL-2 (FIG. 11B). Thus, the cKIT costimulatory constructs demonstrated successful effector-cytokine secretion.

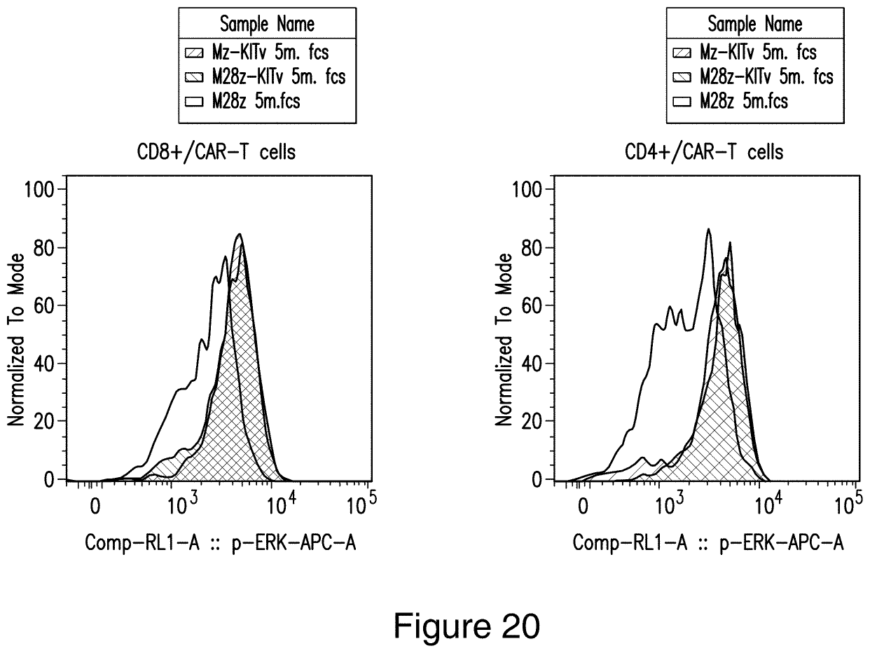

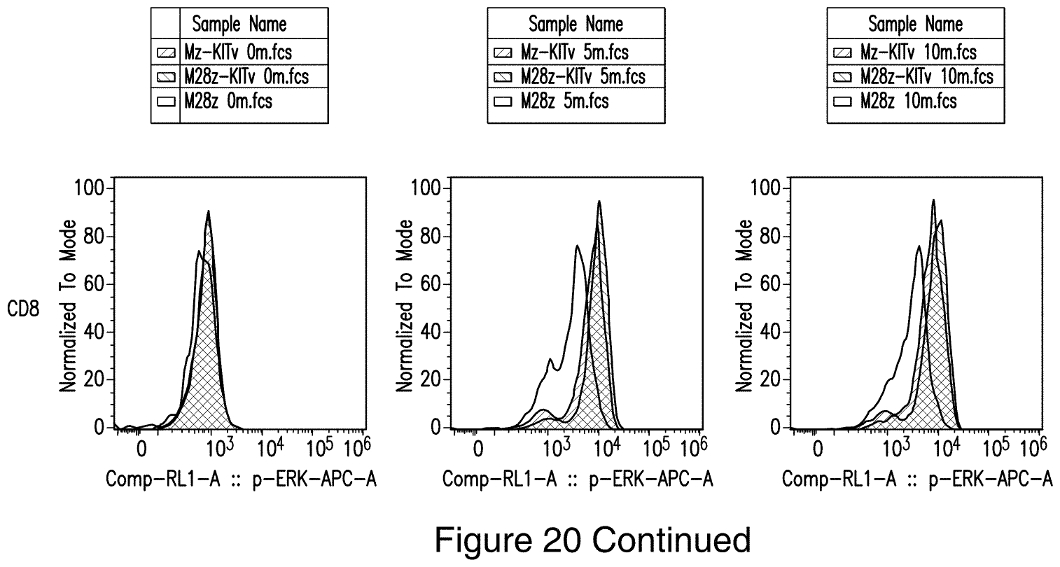

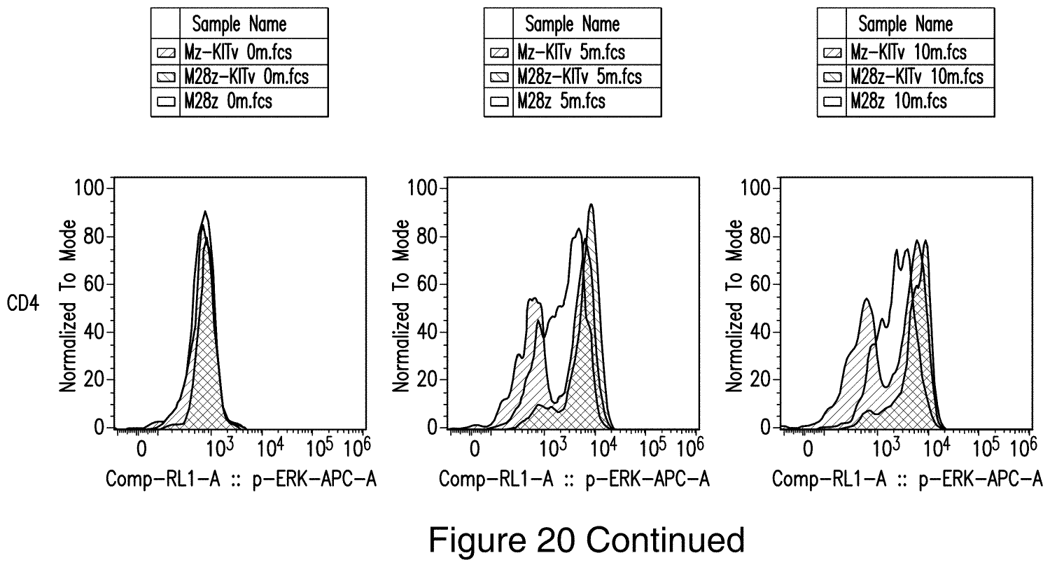

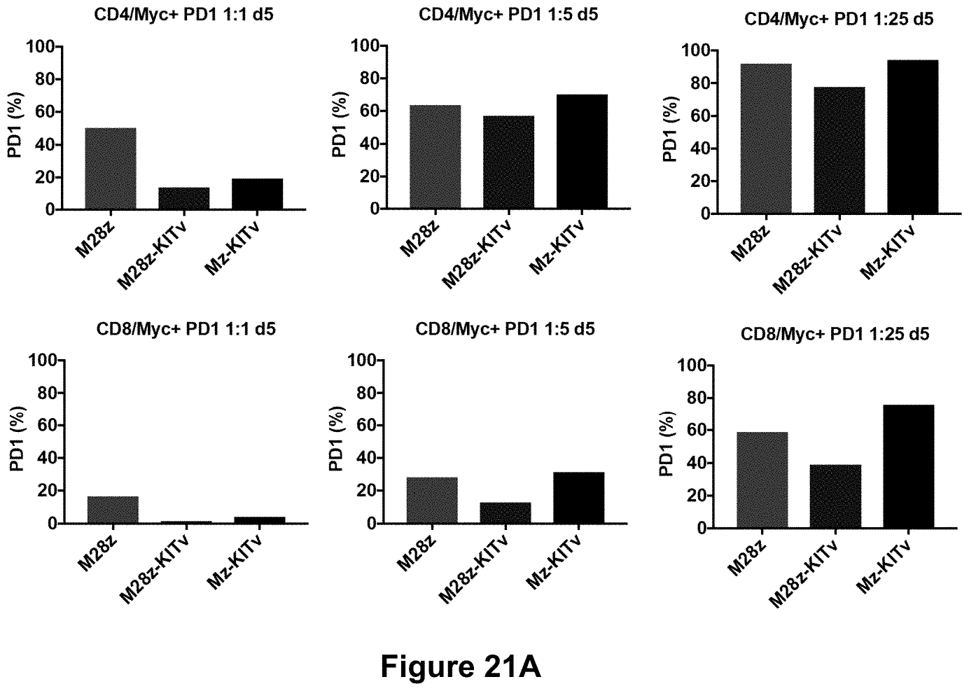

P-ERK signal of CART cells after antigen stimulation was also examined. After co-culture with MGM cell (E:T=1:2) for 5 minutes, p-ERK levels of CD4+ and CD8+ CART cell were measured by FACS. Both CD4 and CD8 CART cells of M28z-KITv and Mz-KITv had stronger p-ERK activity than M28z (FIG. 20).

### Example 5—In Vivo Activities of the Presently Disclosed Constructs

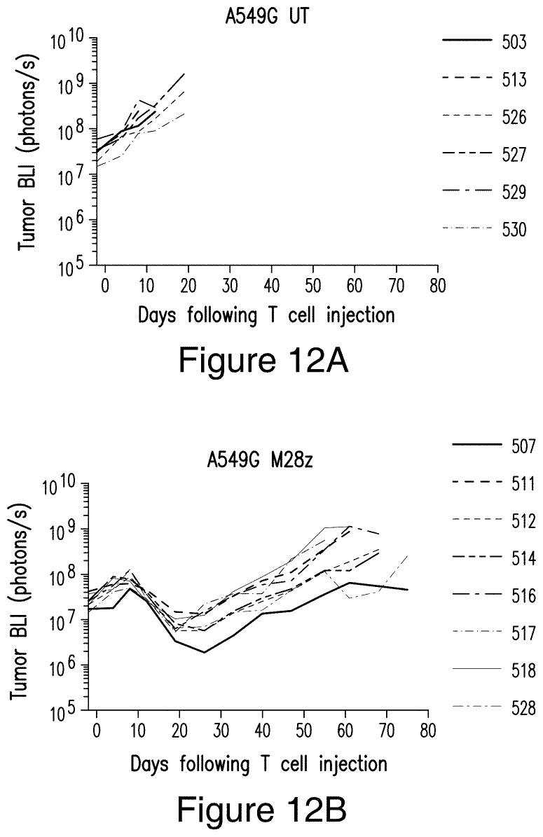

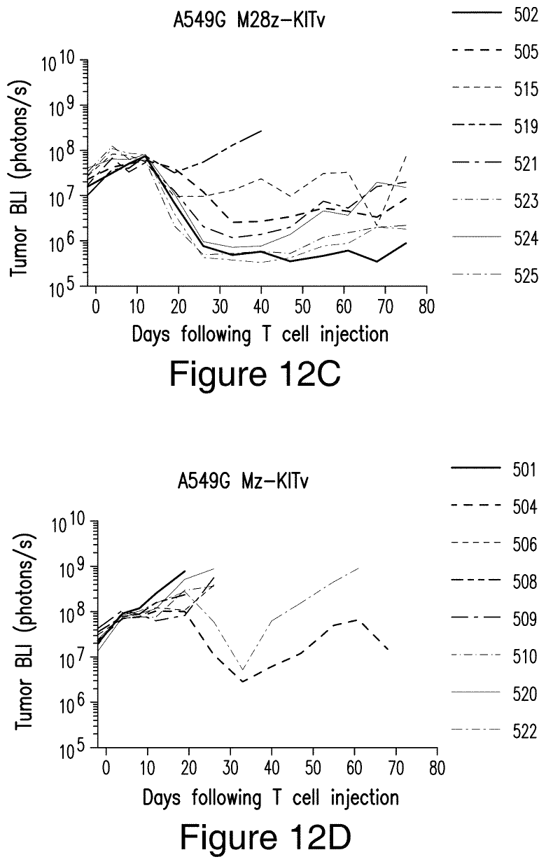

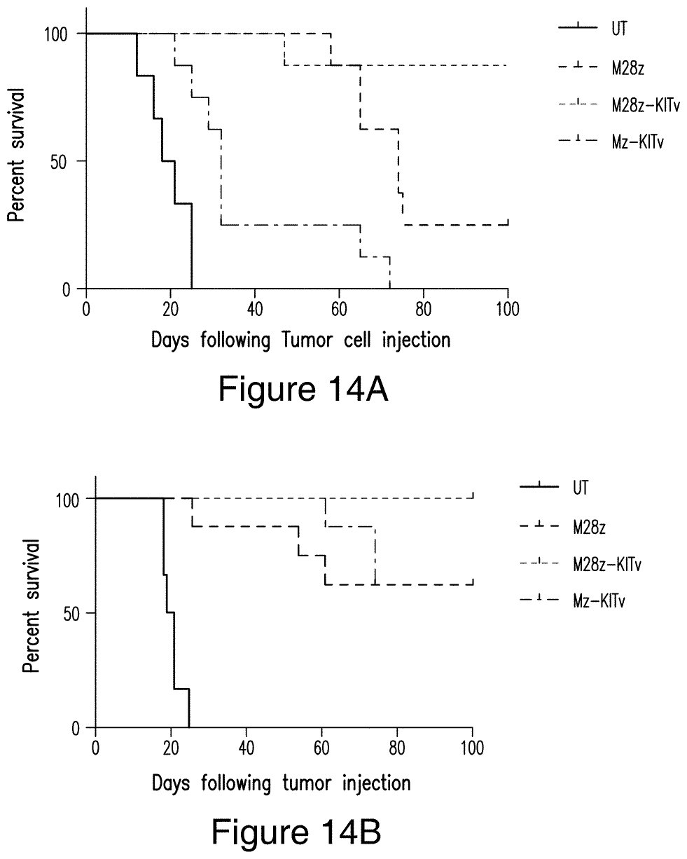

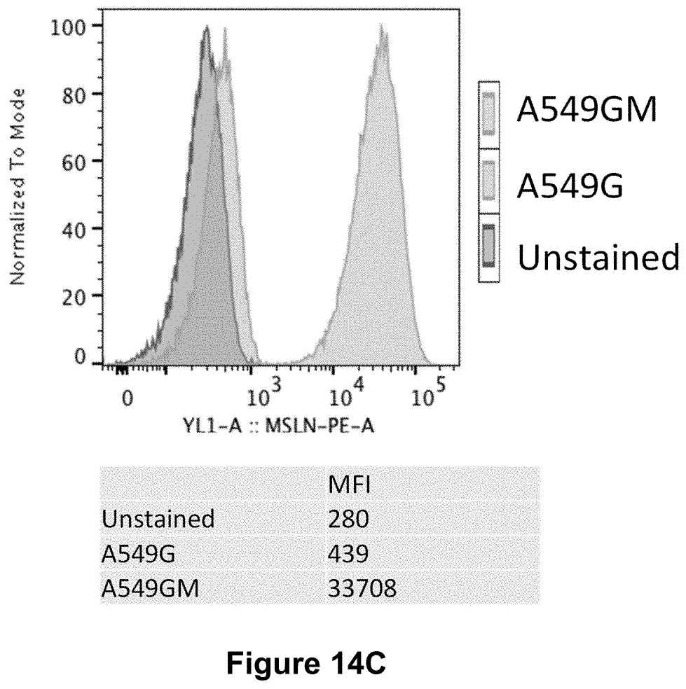

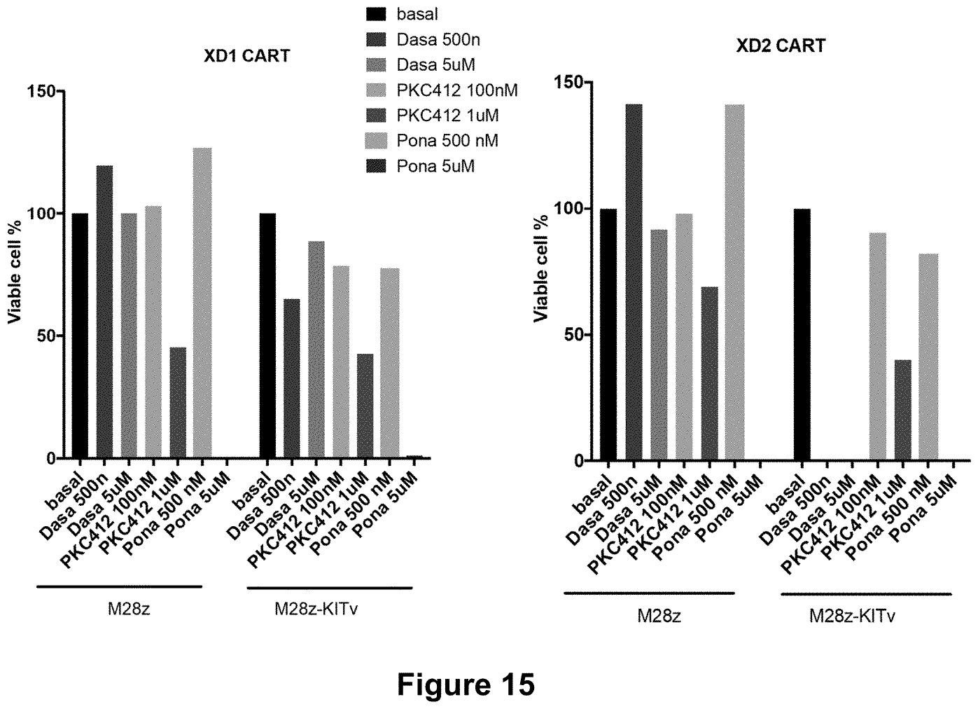

Low mesothelin-expressing lung cancer cells (A549G) and high mesothelin-expressing lung cancer cells (A549GM) were used in establishing lung tumors in NSG mice (FIG. 14C). Mice with established low MSLN A549G lung tumor were treated with a single dose of 1×10⁵ M28z, M28z-KITv and Mz-KITv CART cells. In vivo BLI was used to monitor tumor burden in NSG mice. The results are shown in FIG. 12A-12D. As shown in FIGS. 12A-12D, while treating mice with tumor burden resulting from cancer cells with low-antigen (mesothelin) expression, M28z-KITv CAR T-cell treated mice showed better and long-lasting tumor regression compared to mice treated with M28z CAR T cells or Mz-KITv CAR T cells or untransduced T cells (UT). The mice survival data are presented as Kaplan-meier survival curves in FIG. 14A. As shown in FIG. 14A, while treating mice with low-antigen (mesothelin) expressing tumors, best survival was achieved in mice treated with a single dose of M28z-KITv CAR T cells, e.g., mice treated with M28z-KITv CAR T cells showed prolonged survival as compared to mice treated with M28z CAR T cells.

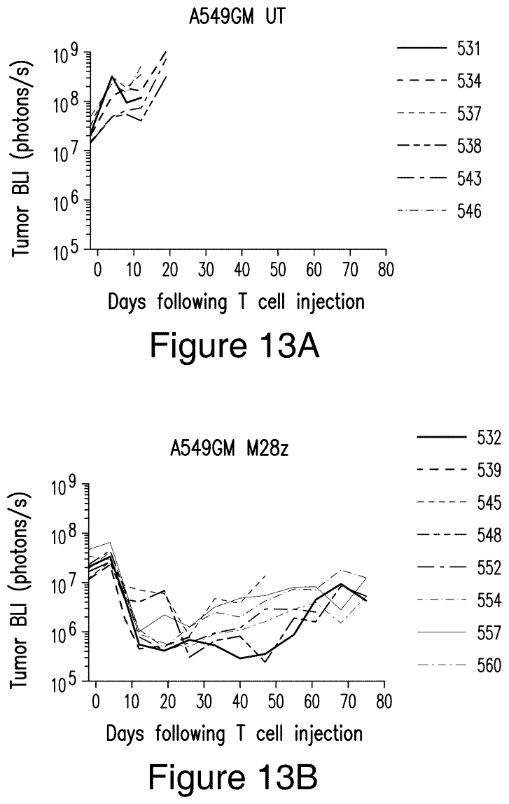

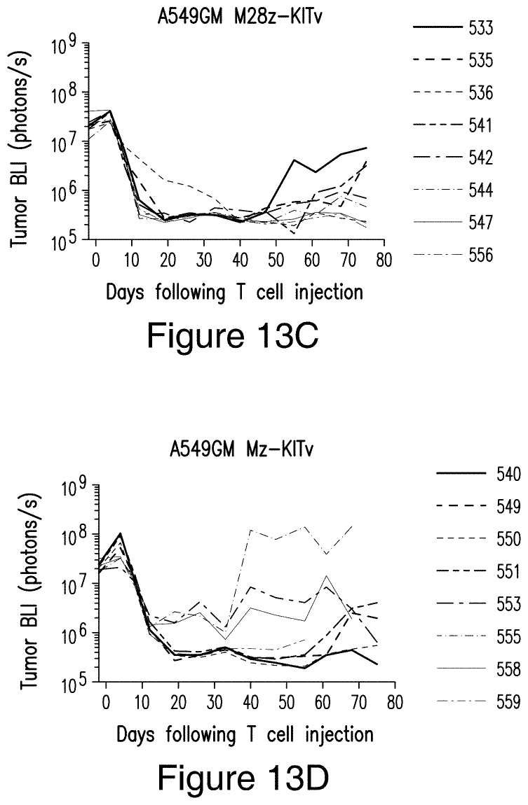

Mice with established high MSLN A549GM lung tumor were treated with a single dose of 1×10⁵ M28z, M28z-KITv and Mz-KITv CAR T cells. In vivo BLI was used to monitor tumor burden in NSG mice. The results are shown in FIG. 13A-13D. As shown in FIGS. 13A-13D, while treating mice with tumor burden resulting from cancer cells with high-antigen (mesothelin) expression, M28z-KITv CAR T-cell treated mice showed better and long-lasting tumor eradication. In addition, Mz-KITv CAR T-cell treated mice showed long-lasting tumor regression equivalent to mice treated with M28z CAR T cells. Mice treated with any of the mesothelin CAR T cells showed impressive tumor regressions compared to mice treated with untransduced (UT) T cells. The mice survival data are presented as Kaplan-meier survival curves in FIG. 14B. As shown in FIG. 14B, while treating mice with high-antigen (mesothelin) expressing tumors, best survival was achieved in mice treated with a single dose of either M28z-KITv or Mz-KITv CAR T cells when compared to mice treated with M28z CAR T cells. In all the 3 groups of mice, median survival was not reached compared to mice treated with untransduced (UT) T cells.

### Example 6—Sensitivity to Tyrosine Kinase Inhibitors of the Constructs

After 7 days stimulation with A549GM cells (E:T=3:1), CART were treated with 500 nM or 5 μM of Dasatinib (Dasa), 500 nM or 5 μM of Ponatinib (Pona), or 100 nM or 1 μM of PKC412 for 72 hours, then viable cells were measured by FACS. As shown in FIG. 15, M28z-KITv CART were more sensitive to tyrosine kinase inhibitors than M28z CAR T cells.

### Example 7—In Vivo Activities of the Presently Disclosed Constructs

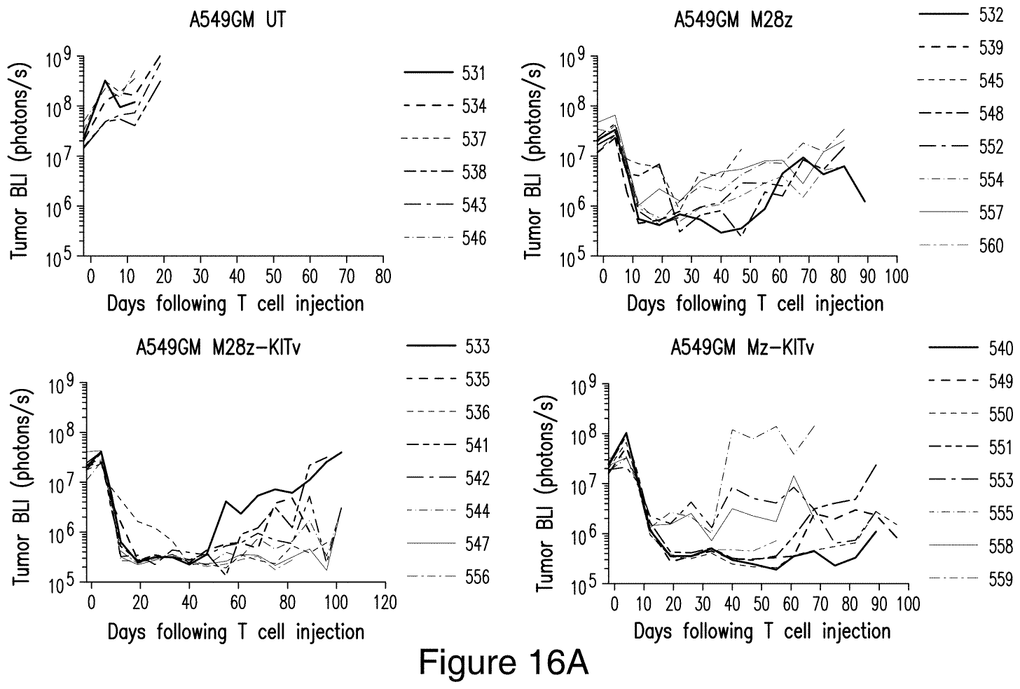

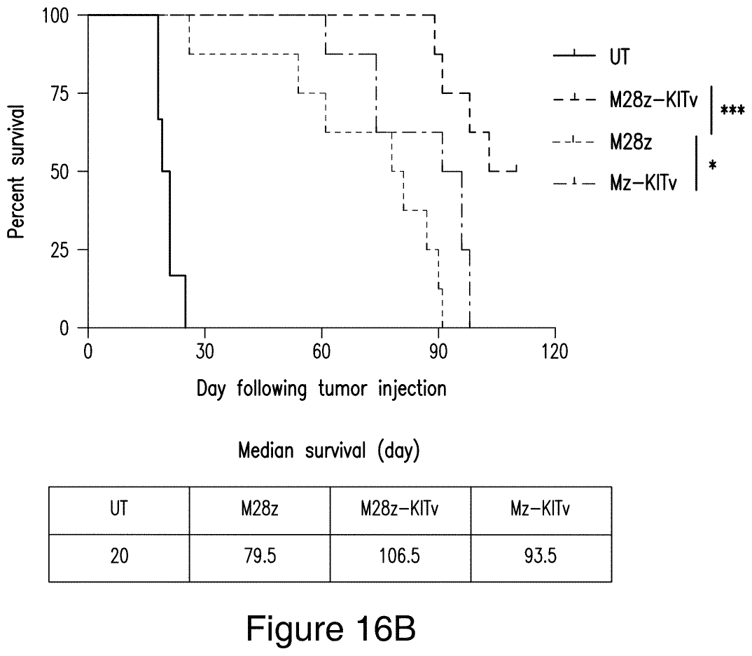

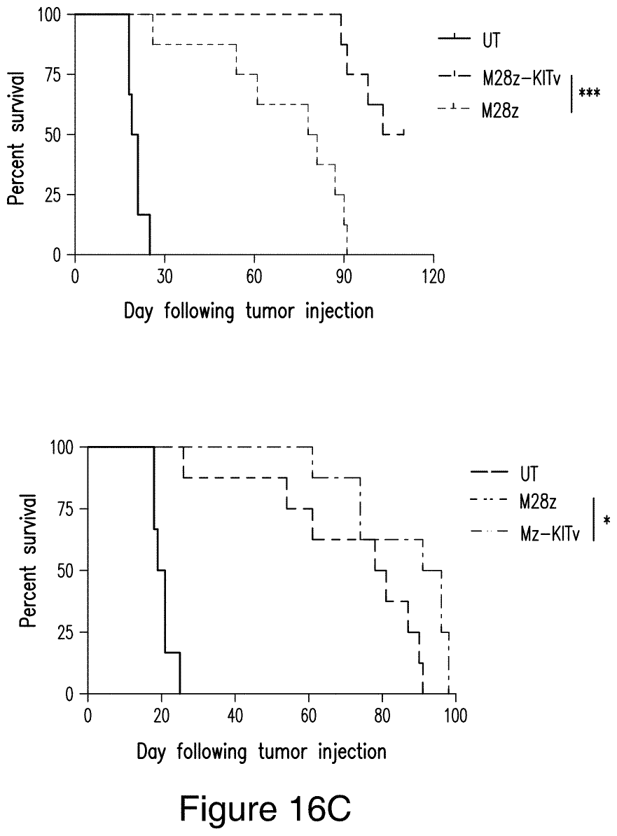

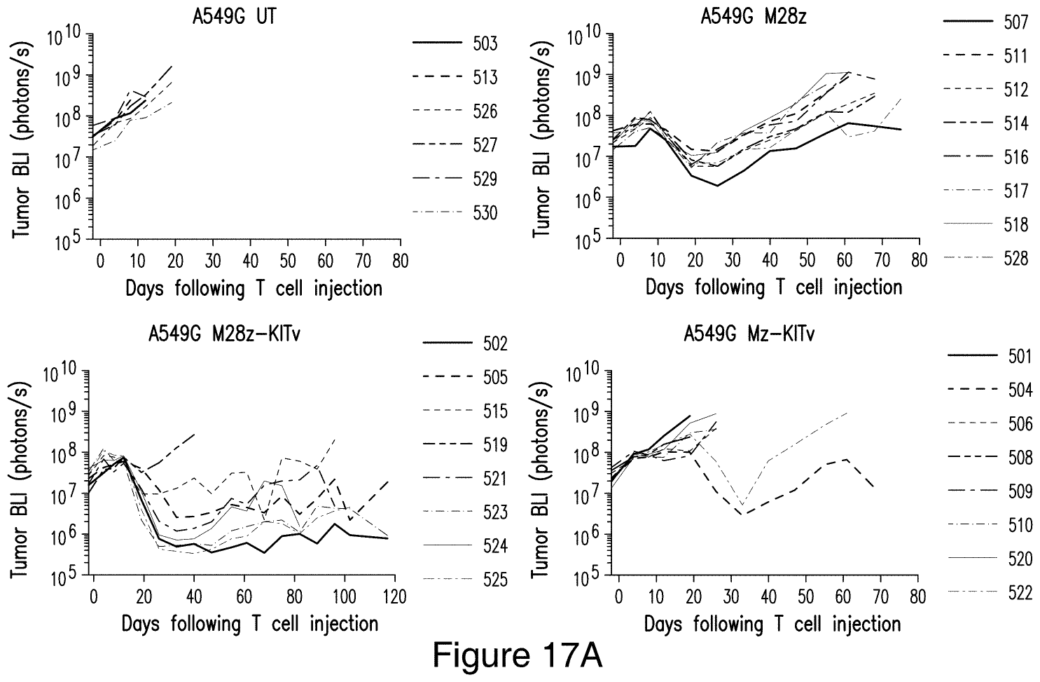

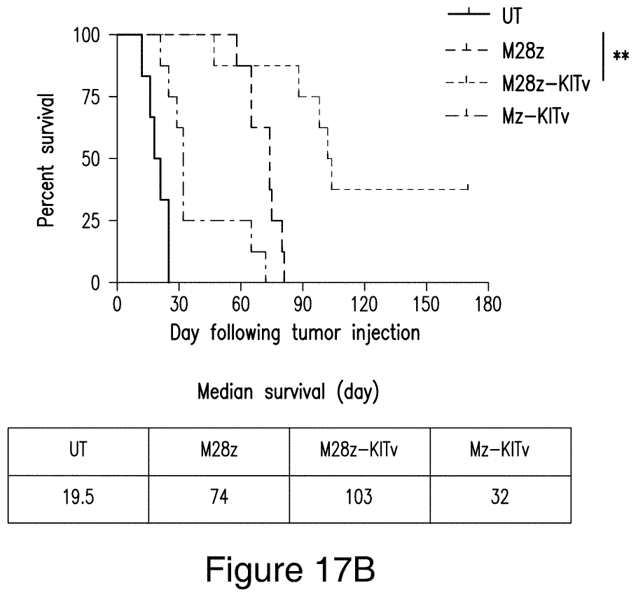

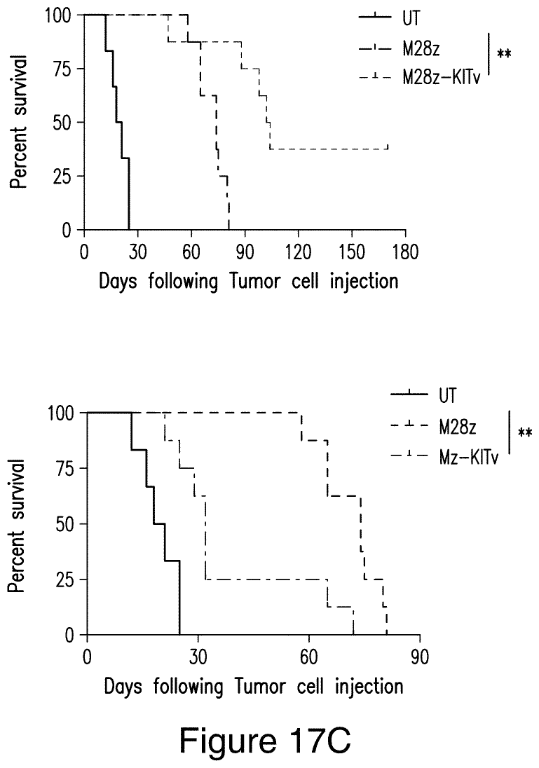

Lung tumor was established in NSG mice using A549GM tumor cells, which had high expression level of mesothelin (MSLN), or A549G tumor cells, which had low expression level of mesothelin (MSLN). The mice were then treated with a single dose of 1×10⁵ M28z, M28z-KITv or Mz-KITv CART cells. Both Mz-KITv and M28z-KITv CAR T cells had higher anti-tumor efficacy against high mesothelin expressing lung cancer cells than M28z CART cells (FIGS. 16A-16C). In low MSLN A549G lung cancer, M28z-KITv CAR T cells had enhanced antitumor activity as compared to M28z CAR T cells, although Mz-KITv CAR T cells had less antitumor activity than M28z CAR T cells (FIGS. 17A-17C).

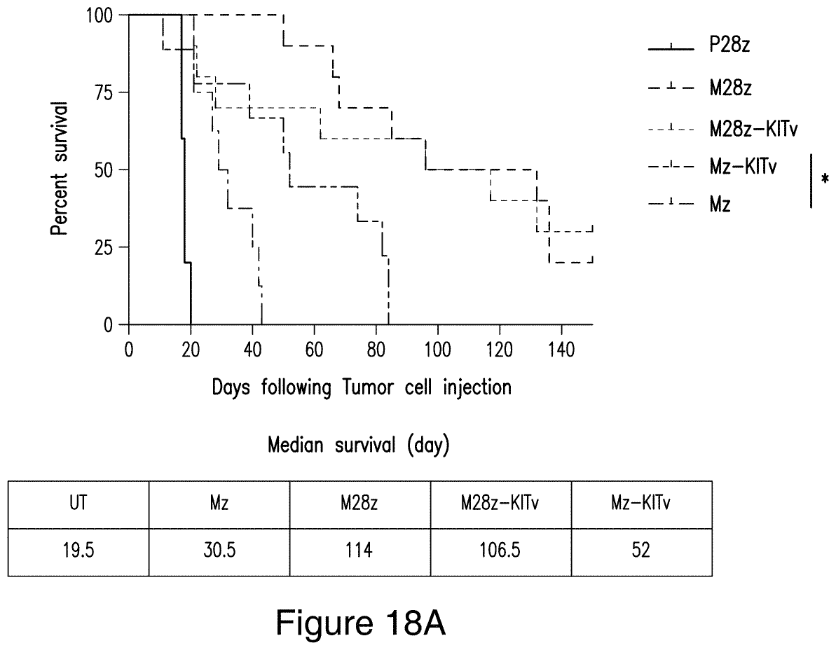

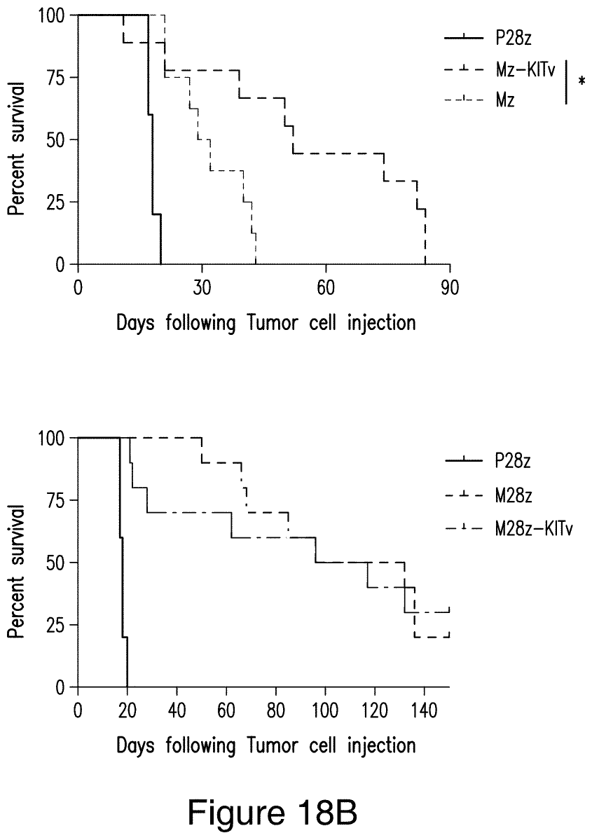

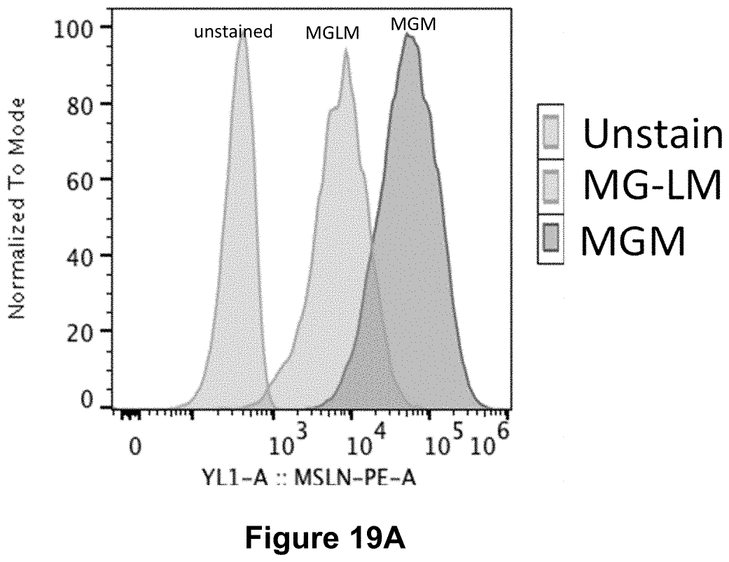

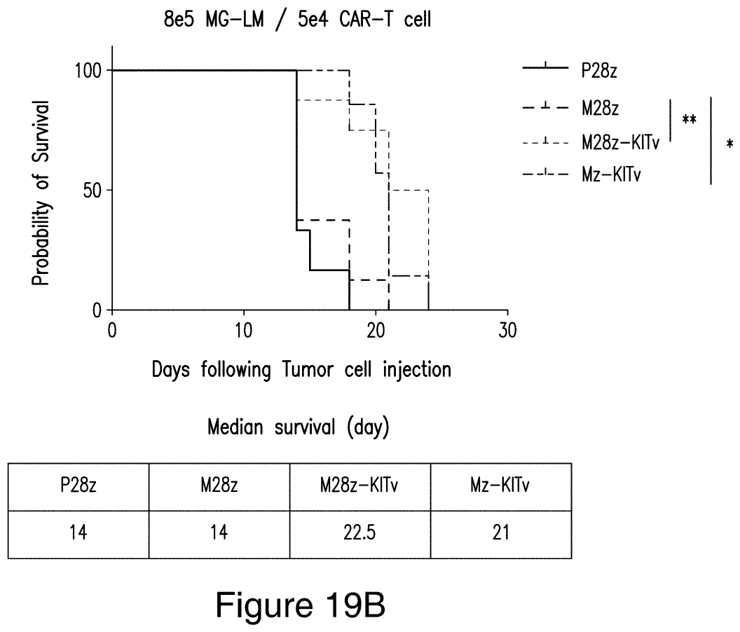

MSTO cell were overexpressed with low and high MSLN protein to produce MG-LM and MGM cell respectively (FIG. 19A). In pleural mesothelioma tumor model, mesothelioma was established in NSG mice through pleural injection of high MSLN-expressing mesothelioma (MGM) tumor cells or low MSLN-expressing mesothelioma (MG-LM) tumor cells. These mice were then intrapleural administered with a single dose of 5×10⁴ P28z, Mz, M28z, M28z-KITv or Mz-KITv CAR T cells. In high MSLN-expressing mesothelioma, Mz-KITv CAR T cells had increased antitumor activity than Mz CAR T cells, although no significant difference between M28z-KITv and M28z CART cells was detected (FIGS. 18A and 18B). CAR T-cell doses were purposefully reduced to mimic low E:T ratios seen in clinic. The reduced cKIT CAR T-cell dose resulted in equivalent anti-tumor efficacy as compared to CD28 CAR T cells. This may be due to PDL1/PD1 pathway related exhaustion at very low E:T ratio. M28z-KITv CAR T cells also had enhanced antitumor activity to low-MSLN expressing mesothelioma (FIG. 19B).

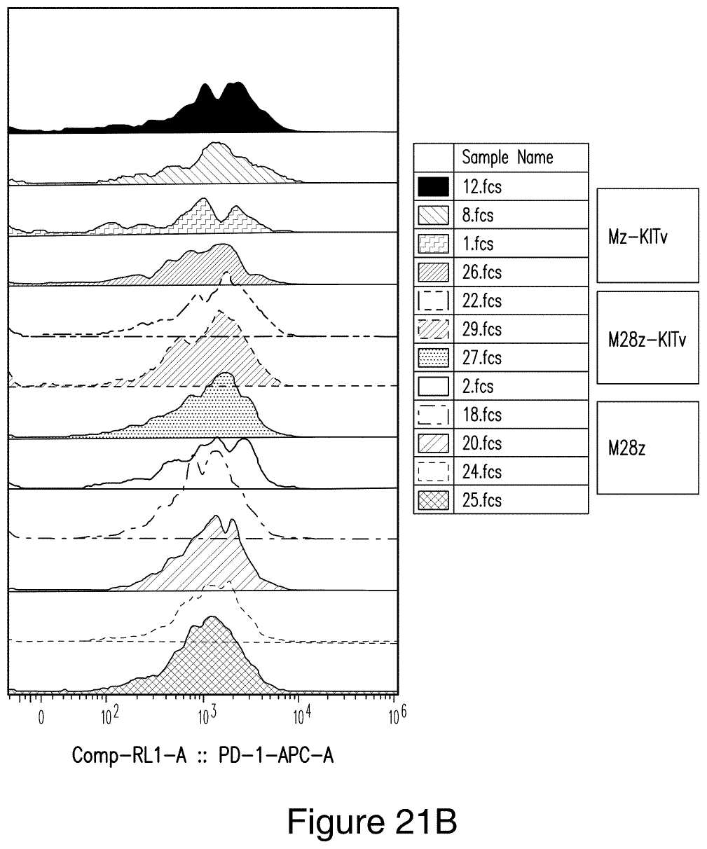

CAR T cells obtained from mice were exposed to high mesothelin-expressing mesothelioma cells and were analyzed for PD1 expression. PD1 upregulation was low at high E:T ratios, but similar at low E:T ratios (FIGS. 21A and 21B).

### Example 8: In Vitro Nano String Analysis

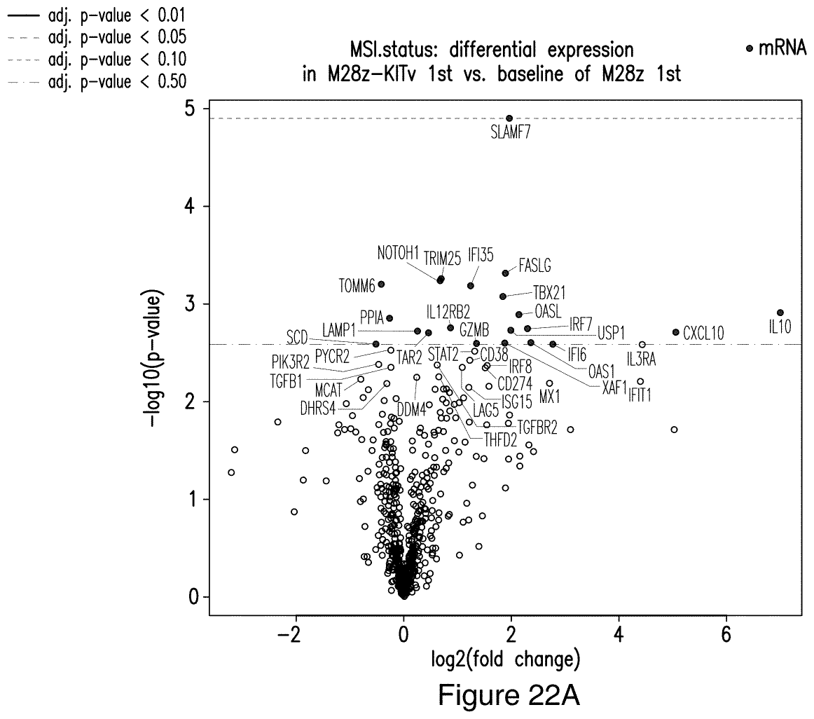

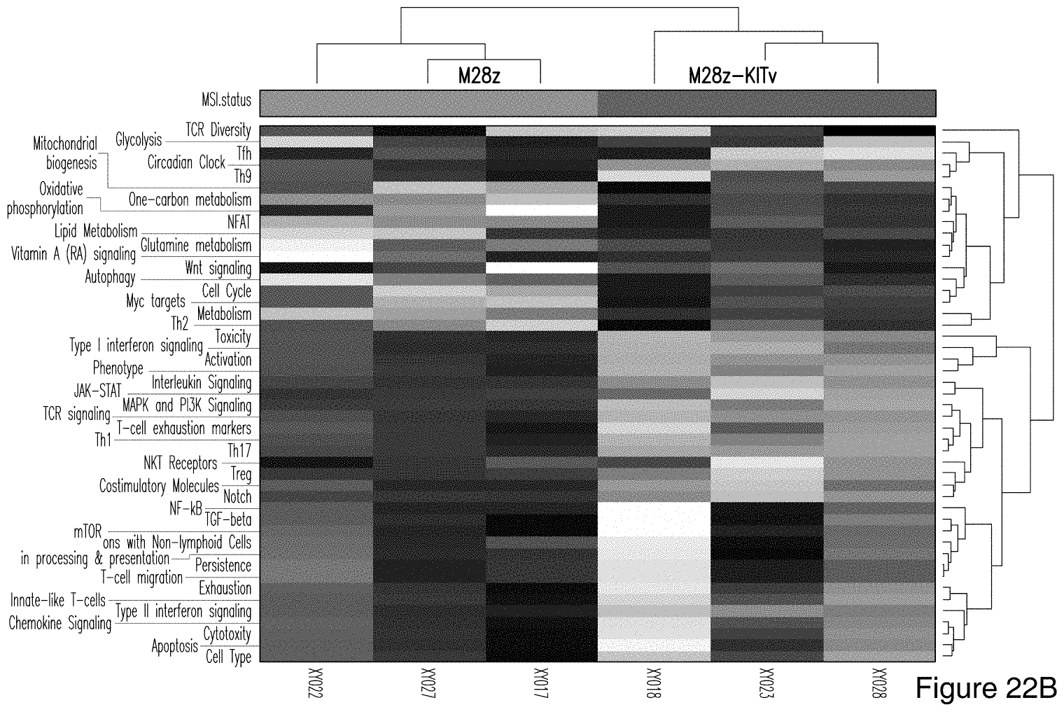

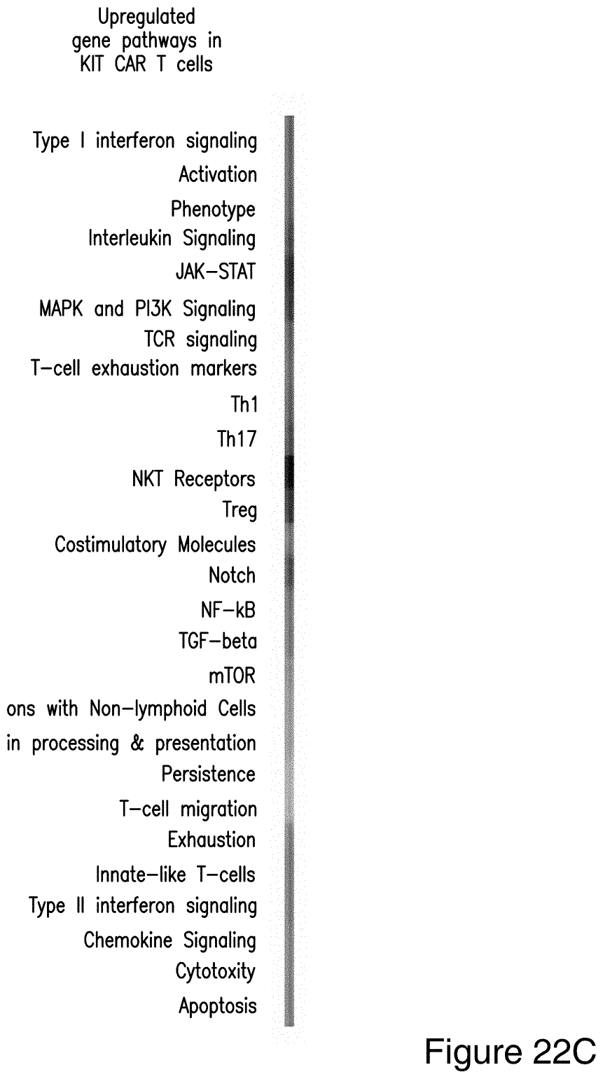

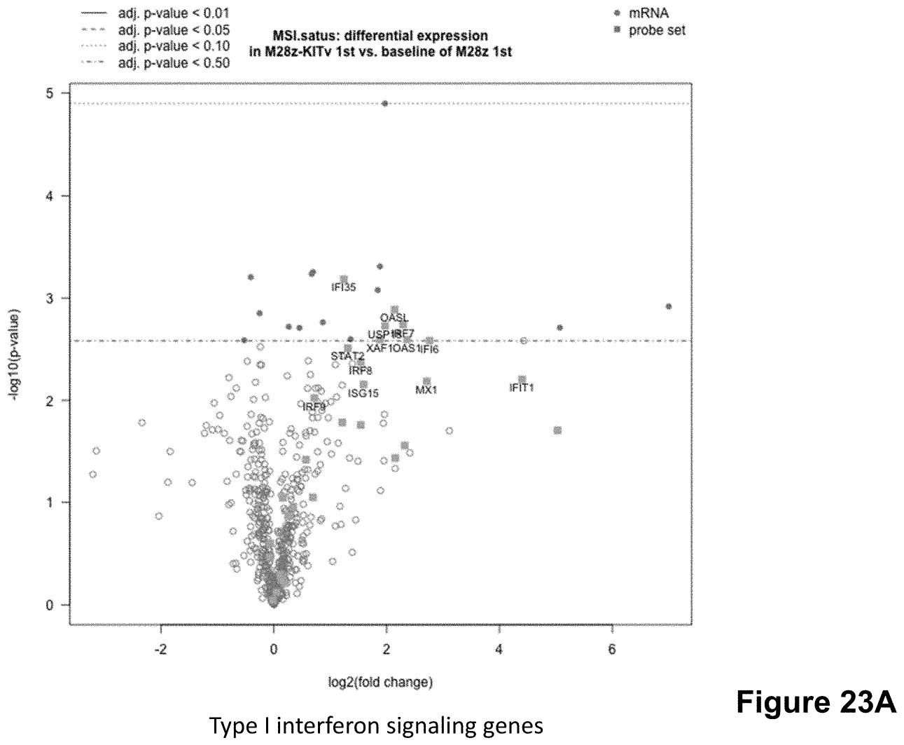

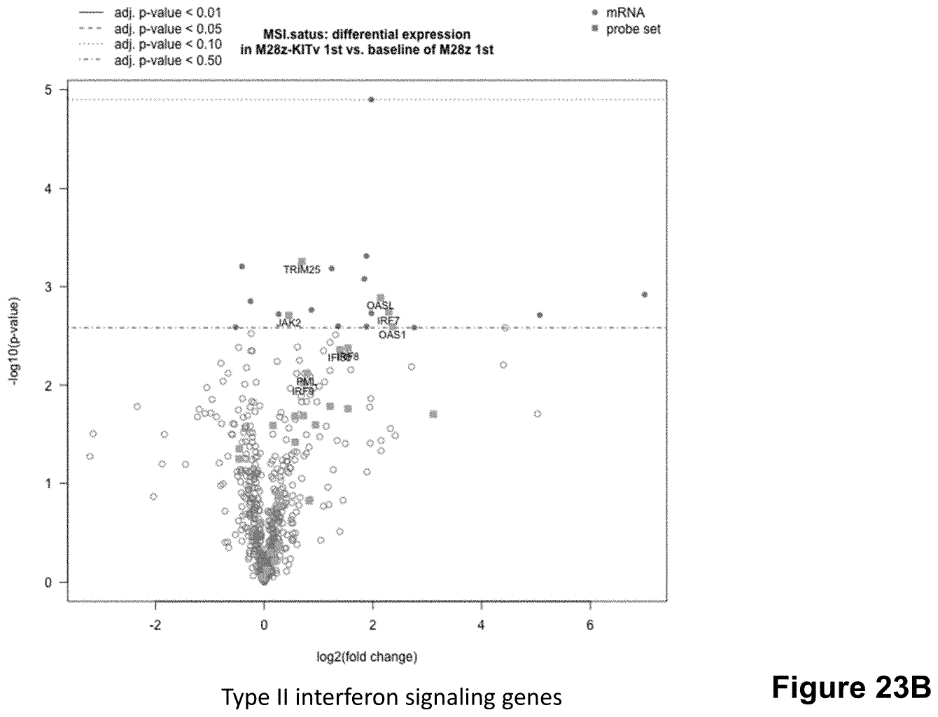

After co-cultured with MSLN⁺ tumor cells for 24 hours, CD8⁺ M28z and M28z-KITv CAR T cells were collected for nano string analysis of CAR T panel genes. 87 out of 780 detected genes had significant fold changes (FIG. 22A). Heatmap of Pathway scores showed enriched gene sets relating to phenotypic and functional T cell features in M28z-KITv CAR T cells (FIG. 22B). Upregulated gene pathways in M28z-KITv CAR T cells were listed in FIG. 22C. The expression of Type I interferon signaling genes (FIG. 23A) and Type II interferon signaling genes (FIG. 23B) significantly increased in M28z-KITv CAR T cells.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A cell comprising:
(a) an antigen-recognizing receptor that binds to an antigen, and
(b) a mutant of c-Kit comprising an activating mutation.

2. (canceled)

3. The cell of claim 1, wherein the activating mutation:
(i) is within the intracellular region of human c-Kit;
(ii) is within amino acids 816 to 826 of human c-Kit;
(iii) is at amino acid position 816 or amino acid position 822;
(iv) is within amino acids 550 to 570 of human c-Kit;
(v) is at amino acid position 560 of human c-Kit;
(vi) is selected from D816V, D816Y, D816H, D816F, N822K, V560G, or a combination thereof; and/or
(vii) comprises or consists of D816V.

4-11. (canceled)

12. The cell of claim 1, wherein the mutant of c-Kit comprises or consists of the amino acid sequence set forth in SEQ ID NO: 2 or a portion thereof.

13-18. (canceled)

19. The cell of claim 1, wherein said antigen-recognizing receptor is recombinantly expressed.

20-21. (canceled)

22. The cell of claim 1, wherein the cell is an immunoresponsive cell.

23-26. (canceled)

27. The cell of claim 1, wherein the antigen is a tumor antigen or a pathogen antigen.

28. (canceled)

29. The cell of claim 27, wherein the tumor antigen is selected from the group consisting of mesothelin, CD19, MUC16, MUC1, CAIX, CEA, CD8, CD7, CD10, CD20, CD22, CD30, CLL1, CD33, CD34, CD38, CD41, CD44, CD49f, CD56, CD74, CD133, CD138, EGP-2, EGP-40, EpCAM, Erb-B2, erb-B3, Erb-B4, FBP, Fetal acetylcholine receptor, folate receptor-a, GD2, GD3, HER-2, hTERT, IL-13R-a2, K-light chain, KDR, LeY, L1 cell adhesion molecule, MAGE-A1, ERBB2, MAGEA3, CT83 (also known as KK-LC-1), p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, CD99, CD70, ADGRE2, CCR1, LILRB2, PRAME, HPV E6 oncoprotein, HPV E7 oncoprotein, and ERBB.

30-31. (canceled)

32. The cell of claim 1, wherein the antigen-recognizing receptor is a CAR.

33-36. (canceled)

37. A method for producing an antigen-specific immunoresponsive cell, the method comprising introducing into a cell (a) a first nucleic acid sequence encoding an antigen-recognizing receptor that binds to an antigen; and (b) a second nucleic sequence encoding a c-Kit mutant comprising an activating mutation.

38-41. (canceled)

42. A composition comprising: a) a mutant of human c-Kit comprising an activating mutation; and b) an antigen-recognizing receptor that binds to an antigen.

43-46. (canceled)

47. The composition of claim 42, wherein the composition is a nucleic acid composition comprising (a) a first polynucleotide encoding the antigen-recognizing receptor and (b) a second polynucleotide encoding the mutant of human c-Kit comprising an activating mutation.

48-53. (canceled)

54. A vector comprising the nucleic acid composition of claim 47.

55. A cell comprising the composition of claim 47.

56. A pharmaceutical composition comprising a cell of claim 1, and a pharmaceutically acceptable excipient.

57. The pharmaceutical composition of claim 56, further comprising an inhibitor of c-Kit.

58-59. (canceled)

60. A method of reducing tumor burden in a subject, the method comprising administering to the subject a cell of claim 1 or a pharmaceutical composition comprising the cell.

61. (canceled)

62. A method of treating and/or preventing a neoplasm, the method comprising administering to the subject a cell of claim 1 or a pharmaceutical composition comprising the cell.

63. A method of lengthening survival of a subject having a neoplasm, the method comprising administering to the subject a cell of claim 1 or a pharmaceutical composition comprising the cell.

64. The method of claim 62, wherein the tumor or neoplasm is a solid tumor.

65-69. (canceled)

70. A kit comprising a cell of claim 1 or a pharmaceutical composition comprising the cell.

71. (canceled)
